(19) United States
(12) Patent Application Publication
(10) Pub. No.: US 2014/0276362 A1
(43) Pub. Date: Sep. 18, 2014

(54) APPARATUS AND METHOD FOR TISSUE REJUVENATION

(71) Applicant: PLUM SYSTEMS, CO., New York, NY (US)

(72) Inventor: Alexandra Alvarez, New York, NY (US)

(73) Assignee: PLUM SYSTEMS,CO., New York, NY (US)

(21) Appl. No.: 14/278,460

(22) Filed: May 15, 2014

Related U.S. Application Data
(63) Continuation of application No. 13/833,902, filed on Mar. 15, 2013.

(51) Int. Cl. A61K 35/12; A61K 33/00; A61M 5/158

(52) U.S. Cl. CPC A61K 35/12; A61M 5/158; A61K 33/00; A61M 2005/006
USPC 604/21; 604/20; 604/23; 424/93.7

(57) ABSTRACT

A method for treating skin in a subject is disclosed. The method comprises the steps of administering a stem cell composition into a target body site in the subject by subcutaneous, intradermal or topical delivery, and contacting the target body site with a treatment device designed for introducing CO₂ into subcutaneous tissue, wherein the CO₂ and the stem cell composition are provided in an amount effective for treating a skin-related disorder or improving the condition of skin. Also disclosed is a method for generating induced pluripotent stem cells (iPSCs) in a subject.

This application is a continuation-in-part application of U.S. patent application Ser. No. 13/833,902, filed on Mar. 15, 2013. The entirety of the aforementioned application incorporated herein by reference in its entirety.

### FIELD

This application generally relates to tissue repair in human subjects. In particular, the invention relates to devices and methods for localized administration of carbon dioxide in combination with a stem cell composition.

### BACKGROUND

Localized subcutaneous administration of carbon dioxide (CO₂), also known as carbon dioxide therapy (CDT) or carboxytherapy is a safe and effective therapy that can improve the appearance of treated skin. Outside of the US, subcutaneous CO₂ administration has found applications in the reduction of skin irregularities, wrinkle reduction and as a complementary treatment to other forms of aesthetic and therapeutic treatments, such as liposuction.

As currently applied, subcutaneous CO₂ administration is a non-surgical method, whereby CO₂ is administered into the subcutaneous tissues through small needles. From the injection point, the CO₂ diffuses into adjacent tissues. While subcutaneous CO₂ administration uses innocuous amounts of inert CO₂ gas, the use of needles as the mode of application requires that a medical professional apply the treatment.

Stem cells are cells having the ability to self-renew and divide to an unlimited extent and to differentiate under suitable circumstances to form different types of cells. Embryonic stem cells (ES cells) are stem cells established from early embryos which can be cultured over a long period of time while maintaining pluripotent ability to differentiate into all kinds of cells existing in living bodies. By contrast, somatic stem cells are any cell which is found in a developed organism that has the ability to divide and create another cell like itself and also divide and create a cell more differentiated than itself.

Thomson et al. (U.S. Pat. No. 5,843,780; Proc. Natl. Acad. Sci. USA 92:7844, 1995) were the first to successfully isolate and propagate pluripotent stem cells from primates. They subsequently derived human embryonic stem (hES) cell lines from human blastocysts (Science 282:114, 1998). Gearhart and coworkers derived human embryonic germ (hEG) cell lines from fetal gonadal tissue (Shamblott et al., Proc. Natl. Acad. Sci. USA 95:13726, 1998; and U.S. Pat. No. 6,090,622 to Gearhart et al.). Both hES and hEG cells have the long-sought characteristics of pluripotent stem cells, i.e., they can be cultured extensively without differentiating, they have a normal karyotype, and they remain capable of producing a number of important cell types.

Induced pluripotent stem (iPS) cells are a type of pluripotent stem cell artificially derived from a non-pluripotent cell, typically an adult somatic cell. Yamanaka et al. transfected mouse fibroblasts with four genes (Oct4, Sox2, c-Myc, Klf4) to obtain iPS cells in 2006. Subsequently, iPS cells were created from human adult somatic cells. (Takahashi et al. Cell, 131:861-872, 2007; Yu et al. Science, 318:1917-1920, 2007). iPS cells derived from differentiated somatic cells having a narrower differentiation profile. iPS cells are capable of differentiating into all of the various cell types in a human body (i.e., pluripotency) and are capable of maintaining semipermanent proliferation while also retaining the karyotype. In addition, iPSC cells express a similar group of gene products that are expressed in ES cells. Hence, the iPSC cells are cells artificially induced by reprogramming of somatic cells, differing from those of differentiated somatic cells, but capable of regenerating differentiated cells ex vivo or in vivo. Stem cell therapies have been proposed for treating a wide variety of disease conditions.

There is a need for improved skin treatment compositions and methods. The present inventors have addressed this need by providing compositions and methods exploiting the benefits of subcutaneous CO₂ administration in combination with stem cell methodologies for improving the treatment of dermatological conditions and for rejuvenating and improving the condition of skin.

### SUMMARY

One aspect of the present application relates to a method of treating skin. The method comprises administering a stem cell composition into a target body site in the subject by subcutaneous, intradermal or topical delivery, and administering CO₂ to the target body site by subcutaneous or intradermal administration, wherein the CO₂ and the stem cell composition are provided in an amount effective for treating a skin-related or rejuvenating skin. The stem cell composition may include a stem cell preparation, a composition comprising one or more iPSC inducing agents or conditioned media obtained from a stem cell culture.

In one embodiment, the stem cell composition comprises one or more hematopoietic stem cells, embryonic stem cells, bone marrow-derived stem cells, mesenchymal stem cells, and/or adipose-derived stem cell. In another embodiment, the stem cell composition comprises induced pluripotent stem cells (iPSCs). The iPSCs may be derived from fibroblasts, skin cells, adipose cells and blood cells, including cells isolated from peripheral blood, bone marrow and umbilical cord blood.

In another embodiment, the stem cell composition includes one or more iPSC inducing agents. In one embodiment the one or more iPSC inducing agents include one or more reprogramming factors selected from the group consisting of Oct 4, Sox2, Klf4, c-Myc, Lin28 and Nanog. In other embodiments, the one or more iPSC inducing agents include one or more members that increase the expression of a reprogramming factor selected from the group consisting of Oct 4, Sox2, Klf4, c-Myc, Lin28 and Nanog. In other embodiments, the iPSC inducing agents include one or more members selected from the group consisting of H3K9 methylation inhibitors, H3K demethylation promotors, HDAC inhibitors, L-type Ca channel agonists, cAMP pathway activators, DNA methyltransferase (DNMT) inhibitors; nuclear receptor ligands, GSK3 inhibitors, MEK inhibitors, TGFβ receptor/ALK5 inhibitors, Erk inhibitors and combinations thereof.

In another embodiment, the stem cell composition includes cell-free components secreted and recovered from a stem cell culture, whereby stem cells in the stem cell culture are selected from the group consisting of embryonic stem cells, iPSCs, hematopoietic stem cells, bone marrow-derived stem cells, mesenchymal stem cells and adipose-derived stem cells.

In another embodiment, the stem cell composition comprises a stem cell extract prepared from a stem cell culture, whereby the stem cell is selected from the group consisting of embryonic stem cells, iPSCs, hematopoietic stem cells, bone marrow-derived stem cells, mesenchymal stem cells and adipose-derived stem cells.

In certain embodiments, the skin may be further treated with one more differentiation agent(s) to promote replenishment and regeneration of cellular skin components from endogenous or exogenously introduced stem cells. Exemplary differentiation agents include ascorbic acid, TGFβ1, retinoic acid, bone morphogenetic protein 4 and combinations thereof.

The methods may further comprise additional treatments selected from the group consisting of galvanic treatment, electrical stimulation, heat treatment, light treatment, and addition of therapeutic agents. The additional treatments may be provided concurrently with the CO₂/stem cell composition administration steps. Alternatively, they may be provided prior to or after said CO₂ treatment.

The CO₂ and stem cell compositions may be introduced with a device comprising a plurality of hollow needles having a diameter of about 0.1 mm to about 0.5 mm and a length of about 0.4 mm to about 2.1 mm. In other embodiments, the stem cell composition may be administered topically.

The compositions and methods described herein may be used to treat various skin-related disorders or improve the condition of skin. Exemplary skin-related disorders or diseases for treatment include psoriasis, Reynaud's disease, a necrotising skin infection, alopecia areata and rheumatoid arthritis.

Another aspect of the present application relates to a method for generating induced pluripotent stem cells (iPSCs) in a subject. The method comprises the step of administrating to a subject a CO₂ treatment comprising: (1) contacting a target body surface of the subject with a skin treatment device comprising: a plurality of hollow needles attached to a contact surface of a housing; and a CO₂ source in fluid communication with at least one of the plurality of hollow needles; (2) applying pressure to the housing such that one or more of the plurality of hollow needles penetrate an epidermis or an outermost layer of cell in the target body surface; and (3) applying an effective amount of CO₂ to the subject through the plurality of hollow needles for an effective period of time to generate iPSCs.

Another aspect of the present application relates to a method for treating a skin-related condition in a subject. The method comprises subjecting the subject to CO₂ treatment that comprises the step of (1) contacting a target body surface of the subject with a skin treatment device comprising plurality of hollow needles attached to a contact surface of a housing and a CO₂ source in fluid communication with at least one of said plurality of hollow needles; (2) applying pressure to the housing such that one or more of the plurality of hollow needles penetrate an epidermis or an outermost layer of cell in the target body surface; and (3) applying an effective amount of CO₂ to the subject through the plurality of hollow needles for an effective period of time to generate a low-pH environment adapted to induce reprogramming of somatic cells into induced pluripotent stem cells. In some embodiments, the skin-related condition is selected from the group consisting of psoriasis, Reynaud's disease, a necrotising skin infection, alopecia areata and rheumatoid arthritis.

### BRIEF DESCRIPTION OF THE DRAWINGS

The present invention can be better understood by reference to the following drawings. The drawings are merely exemplary to illustrate certain features that may be used singularly or in combination with other features and the present invention should not be limited to the embodiments shown.

FIG. 1 depicts the exterior contact surface of an exemplary application device.

FIG. 2 shows the interior of the contact surface of FIG. 1.

FIG. 3 shows the valves of the exemplary application device of FIG. 1.

FIG. 4 shows the handle and controls of the exemplary application device of FIG. 1.

FIG. 5 is a side view of the exemplary application device of FIG. 1 showing the CO₂ supply tube.

FIG. 6 shows another exemplary embodiment of an application device having microneedles affixed thereto.

FIG. 7 is an illustration of the penetration of the skin by the microneedles of the device.

FIG. 8 depicts an exemplary configuration for the microneedles in the base element.

FIG. 9 depicts another exemplary configuration for the microneedles in the base element.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description and claims, the singular forms “a,” “an” and “the” include plurals unless the context clearly dictates otherwise. The present invention provides improved methods and devices to apply subcutaneous or intradermal CO₂ administration in combination with one or more stem cell composition components to an area of a patient. The device includes a treatment device fittable with a microneedle/base element where a plurality of the microneedles are in fluid communication with a source of CO₂. To apply the CO₂, the device is placed on the surface of the skin or scalp of the patient as described below

As used herein, “administering to the skin in need of such treatment” means contacting (e.g., by use of the hands or an applicator) the area of skin in need such treatment. These features may be present on the face, such as under or adjacent the eyes, nose, forehead, cheeks, jowls, and neck, as well as other areas of the body such as the arms, chest, back, shoulder, belly (e.g., stretch marks), and legs (e.g., cellulite).

As used herein, the term “pluripotent” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. In addition, non-limiting markers characteristic of human pluripotent stem cells SSEA-3, SSEA-4, TRA-1-60, TRA-1-81, TRA-2-49/6E, ALP, Sox2, E-cadherin, UTF-I, Oct4, Lin28, Rex1, and Nanog.

The term “induced pluripotent stem cell” (iPSC), as used herein, refers to a pluripotent stem cell dedifferentiated from a non-pluripotent cell. The non-pluripotent cell has a reduced potency to self-renew and differentiate than the pluripotent stem cell. Cells of lesser potency can be, but are not limited to, somatic stem cells, tissue specific progenitor cells, primary or secondary cells.

The term “induced pluripotent stem cell inducing agent” refers to small molecules (e.g., chemical compound and molecules with a molecular weight less than 1,000 dalton) or large molecules (e.g., polypeptides and polynucleotides) capable of stimulating the dedifferentiation of non-pluripotent cell, such as a somatic cell, into an induced pluripotent stem cell.

The term “reprogramming” as used herein is also referred to as “nuclear reprogramming” and refers to a process or means during which differentiated somatic cells are induced and converted into undifferentiated pluripotent cells.

The term “stem cell composition” refers to a stem cell preparation, a composition comprising one or more iPSC inducing agents or conditioned media obtained from a stem cell culture. In some embodiments, the stem cell composition is a cell-free composition.

The term “treating” or “treatment” of a skin disorder means the treatment (e.g., complete or partial reduction or elimination of symptoms and/or cure), prevention, amelioration, or inhibition of a skin-related disorder and/or rejuvenation of skin.

As used herein, the term “skin-related disorder” or “skin condition” means a disease, disorder, or defect of the skin. In some embodiments, skin-related disorders include disorders or diseases that affect the condition or appearance of skin, such as Reynaud's disease and rheumatoid arthritis.

One aspect of the present application relates to a method of treating skin. The method comprises administering a stem cell composition into a target body site in the subject by subcutaneous or topical delivery and contacting the target body site with a device designed for introducing CO₂ into subcutaneous tissue, wherein the CO₂ and the stem cell composition are provided in an amount effective for treating a skin-related disorder or improving the condition of skin.

In some embodiments, the CO₂ treatment enhances tissue rejuvenation when administered in combination with the stem cell composition. In particular, the CO₂ treatment may enhance production of induced pluripotent stem cells from endogenous somatic cells or endogenous somatic cells in combination with iPSC inducing agents. Alternatively, the CO₂ treatment may enhance the bioactivity and therapeutic effect of stem cells transplanted into a subject as further described herein.

### Stem Cell Compositions

A stem cell composition in accordance with the present application may include a stem cell preparation, one or more iPSC inducing agents, cell-free components secreted from a stem cell culture, a stem cell extract or a combination thereof.

In some embodiments, the stem cell composition comprises a stem cell selected from the group consisting of hematopoietic stem cells, embryonic stem cells, bone marrow-derived stem cells, mesenchymal stem cells, and adipose-derived stem cells. In another embodiment, the stem cell composition comprises an iPSC produced from a differentiated cell. Preferably, the induced pluripotent cell is produced or reprogrammed ex vivo from a subject's own cells. By way of example, the subject's cells may be derived from adipose cells, skin cells, fibroblasts or blood cells. Exemplary blood cells include cells isolated from peripheral blood, bone marrow and umbilical cord blood.

In other embodiments, the stem cell composition includes one or more iPSC inducing agents. The iPSC inducing agents promote the reprogramming of existing somatic cells in the subject's target site into iPSCs. In some embodiments, iPSC inducing agents include one or more reprogramming factors or agents that can promote reprogramming of somatic cells into iPSCs and/or increase expression of one or more reprogramming factors. Reprogramming factors as used herein may include proteins, nucleic acids, or combinations thereof. Exemplary reprogramming factors include Oct3/4, Sox2, and Klf4; Oct3/4, Klf4, and c-Myc; Oct3/4, Sox2, Klf4, and c-Myc; Oct3/4 and Sox2; Oct3/4, Sox2, and Nanog; Oct3/4, Sox2, and Lin28; and Oct3/4 and Klf4, for example. Reprogramming factors that can be used in the present invention comprise at least Oct3/4 and Nanog. Oct3/4 is also referred to as Oct3, Oct4, or POU5F1, indicating the same transcription factor. Herein, they may be collectively or interchangeably referred to as Oct4.

In some embodiments, reprogramming agents are capable of increasing the expression of one or more genes selected from the group consisting of Oct3/4, Sox2, cMyc, Klf4, Lin28 and Nanog. In some embodiments, the reprogramming agents are plasmid vectors or viral vectors capable of expressing of one or more genes selected from the group consisting of Oct3/4, Sox2, cMyc, Klf4, Lin28 and Nanog. In other embodiments, the reprogramming agents are plasmid vectors or viral vectors capable of stimulating expression of one or more genes selected from the group consisting of Oct3/4, Sox2, cMyc, Klf4, Lin28 and Nanog.

Oct4 is one of the family of octamer (“Oct”) transcription factors, and plays a crucial role in maintaining pluripotency. The removal or neutralization of Oct4 from Oct4+ cells, such as blastomeres and embryonic stem cells, leads to spontaneous trophoblast differentiation. Conversely, the presence of Oct4 thus gives rise to the pluripotency and/or increases differentiation potential of embryonic stem cells. Various other genes in the “Oct” family, including Oct4's close relatives, Oct1 and Oct6, fail to elicit induction, thus demonstrating the exclusiveness of Oct4 to the induction process.

The Sox family of genes is associated with maintaining pluripotency similar to Oct4, although it is associated with multipotent and unipotent stem cells in contrast with Oct4, which is exclusively expressed in pluripotent stem cells. While Sox2 was the initial gene used for induction of pluripotent cells, other genes in the Sox family have been found to work as well in the induction process. Sox1 yields iPS cells with a similar efficiency as Sox2, and genes Sox3, Sox15, and Sox18 also generate iPS cells, although with decreased efficiency.

The Klf family of genes were identified as factors for the generation of iPS cells. Klf2 and Klf4 were found to be factors capable of generating iPS cells, and related genes Klf1 and Klf5 did as well, although with reduced efficiency.

The Myc family of genes are proto-oncogenes implicated in cancer. c-myc is a factor implicated in the generation of mouse and human iPS cells. N-myc and L-myc have been identified to induce pluripotency with an efficiency similar to that of c-myc.

Nanog is a transcription factor critically involved with self-renewal of undifferentiated embryonic stem cells. LIN28 is an mRNA binding protein expressed in embryonic stem cells and embryonic carcinoma cells associated with differentiation and proliferation. Glis1 is a highly promiscuous transcription factor, regulating the expression of numerous genes.

In some embodiments, the iPSC inducing agents include H3K9 histone methylation inhibitors, H3K histone demethylation promotors, histone deacetylase (HDAC) inhibitors, L-type calcium channel agonists, cAMP pathway activators, DNA methyltransferase (DNMT) inhibitors; nuclear receptor ligands, GSK3 inhibitors, MEK inhibitors, TGFβ receptor/ALK5 inhibitors, HDAC inhibitor, Erk inhibitors and combinations thereof. In certain embodiments, the iPSC inducing agents are proteins, peptides, DNA molecules (including antisense), RNA molecules (including RNAi, antisense, miRNAs) or small molecules directed to any of the above targets.

In some embodiments, the stem cell composition comprises an effective amount of a histone deacetylase (HDAC) inhibitor, a histone methyltransferase inhibitor, a calcium channel activator or combination thereof. In another embodiments, the treatment composition comprises an effective amount of a histone methyl transferase inhibitor and an L-type calcium channel agonist.

Exemplary H3K9 methylation inhibitors include methyltransferase inhibitors, including G9a histone methyltransferase inhibitors, such as BIX-01294 and S-adenosyl-methionine (SAM) analogs, such as methylthio-adenosine (MTA), sinefungin and S-adenosyl-homocysteine (SAH), chaetocin, 3-deazaneplanocin A hydrochloride, (R)—PFI 2 hydrochloride, SGC 0496, UNC 0224, UNC 0638, UNC 0642 and UNC 0646.

Exemplary HDAC inhibitors include hydroxamic acids or hydroxamates (such as trichostatin A), cyclic tetrapeptides (such as trapoxin B) and depsipeptides, benzamides, electrophilic ketones and aliphatic acid compounds (such as phenylbutyrate and valproic acid), hydroxamic acids (such as vorinostat, belinostat, LAQ824, panobinostat), benzamides (such as entinostat, CI994, and mocetinostat), abexinostat, SB939, resminostat, givinostat, quisinostat, nicotinamide, CUDC-101, AR-42, CHR-2845, CHR-3996, CG200745, ACY-1215, ME344, MS27-275, AN-9, apicidin derivatives, Baceca, CBHA, CHAPs, chlamydocin, CS-00028, CS-055, EHT-0205, FK-228, FR-135313, G2M-777, HDAC-42, LBH-589, MGCD-0103, NSC-3852, PXD-101, pyroxamide, SAHA derivatives, suberanilohydroxamic acid, tacedinaline, VX-563, MC1568, zebularine, sulforaphane, kevetrin, derivatives of nicotinamide adenine dinucleotide (NAD), dihydrocoumarin, naphthopyranone, 2-hydroxynaphaldehydes, and romidepsin. In one embodiment, the one or more HDAC inhibitor is selected from the group consisting of trichostatin A, MS27-275, and MC1-568. In another embodiment, the HDAC inhibitor is valproic acid.

L-type calcium channel agonists include, but are not limited to, (±)-Bay K 8644, (S)-(−)-Bay K 8644, Dehydrodidemnin, S(+)-PN 202-791, CGP 48506, FPL 64176 and nefiracetam.

Exemplary cAMP pathway agonists include, but are not limited to, forskolin, FSH, milrinone, cilostamide, rolipram, dbcAMP and 8-Br-cAMP. Exemplary DNA methyltransferase (DNMT) inhibitors include antibodies that bind, dominant negative variants of, and siRNA and antisense nucleic acids that suppress expression of DNMT, as well 5-aza-C (5-azacitidine or azacitidine), 5-aza-2′-deoxycytidine (5-aza-CdR), decitabine, doxorubicin, EGCG ((−)-epigallocatechin-3-gallate), RG108 and zebularine.

Nuclear receptor ligands may include agonists, antagonists, activators and/or repressors of nuclear receptors that can modulate local gene expression or transcription at the site of delivery and may regulate epigenetic states of specific gene loci where they bind. Exemplary nuclear receptor ligands include dexamethasone (e.g., at 1 μM, a glucocorticoid receptor agonist), ciglitazone and Fmoc-Leu (both used at e.g., 5 μM) (a PPAR agonist), Bexarotene (e.g., at (3 μM) (a RXR antagonist), estradiol (e.g., 17-beta estradiol), all-trans retinoic acid, 13-cis retinoic acid, dexamethasone, clobetasol, androgens, thyroxine, vitamin D3 glitazones, troglitazone, pioglitazone, rosiglitazone, prostaglandins, and fibrates (e.g., bezafibrate, ciprofibrate, gemfibrozil, fenofibrate and clofibrate).

Exemplary inhibitors of GSK3 may include antibodies that bind, dominant negative variants of, and siRNA and antisense nucleic acids that target GSK3. Specific examples of GSK3 inhibitors include, but are not limited to, Kenpaullone, 1-Azakenpaullone, CHIR99021, CHIR98014, AR-AO14418, CT 99021, CT 20026, SB216763, AR-AO14418, lithium, SB415286, TDZD-8, BIO (2′Z,3′£)-6-Bromoindirubin-3′-oxime (GSK3 Inhibitor IX); BIO-Acetoxime (2′Z,3′E)-6-Bromoindirubin-3′-acetoxime (GSK3 Inhibitor X); (5-Methyl-1H-pyrazol-3-yl)-(2-phenylquinazolin-4-yl)amine (GSK3-Inhibitor XIII); Pyridocarbazole-cyclopenadienylruthenium complex (GSK3 Inhibitor XV); TDZD-8 4-Benzyl-2-methyl-1,2,4-thiadiazolidine-3,5-dione (GSK3beta Inhibitor I); 2-Thio(3-iodobenzyl)-5-(1-pyridyl)-[1,3,4]-oxadiazole (GSK3beta Inhibitor II); OTDZT 2,4-Dibenzyl-5-oxothiadiazolidine-3-thione (GSK3beta Inhibitor III); alpha-4-Dibromoacetophenone (GSK3beta Inhibitor VII); AR-AO 14418 N-(4-Methoxybenzyl)-N′-(5-nitro-1,3-thiazol-2-yl)urea (GSK-3beta Inhibitor VIII); 3-(1-(3-Hydroxypropyl)-1H-pyrrolo[2,3-b]pyridin-3-yl]-4-pyrazin-2-yl-pyrrole-2,5-dione (GSK-3beta Inhibitor XI); TWS 1 19 pyrrolopyrimidine compound (GSK3beta Inhibitor XII); L803 H-KEAPPAPPQSpP-NH2 or its Myristoylated form (GSK3beta Inhibitor XIII); 2-Chloro-1-(4,5-dibromo-thiophen-2-yl)-ethanone (GSK3beta Inhibitor VI); AR-AO144-18; SB216763; and SB415286. Inhibitors of MEK can include antibodies to, dominant negative variants of, and siRNA and antisense nucleic acids that suppress expression of, MEK. Exemplary MEK inhibitors include, but are not limited to, PD0325901, PD98059 (available, e.g., from Cell Signaling Technology), U0126 (available, for example, from Cell Signaling Technology), SL 327 (available, e.g., from Sigma-Aldrich), ARRY-162 (available, e.g., from Array Biopharma), PD184161, PD184352 (CI-1040), sunitinib, sorafenib, vandetanib, pazopanib, axitinib and PTK787. Additional MEK inhibitors may include one or more members undergoing clinical trial evaluations, including CI-1040, PD184352, BAY 43-9006, PD-325901, GSK1120212, ARRY-438162, RDEA119, AZD6244 (also ARRY-142886 or ARRY-886), RO5126766, XL518 and AZD8330 (also ARRY-704).

TGF beta receptor (e.g., ALK5) inhibitors can include antibodies to, dominant negative variants of, and antisense nucleic acids that suppress expression of, TGF beta receptors (e.g., ALK5). Exemplary TGFβ receptor/ALK5 inhibitors include, but are not limited to, SB431542, A-83-01 (also known as 3-(6-Methyl-2-pyridinyl)-N-phenyl-4-(4-quinolinyl)-1H-pyrazole-1-carbothi-oamide), 2-(3-(6-Methylpyridin-2-yl)-1H-pyrazol-4-yl)-1,5-naphthyridine, Wnt3a/BIO, BMP4, GW788388 (-{4-[3-(pyridin-2-yl)-1H-pyrazol-4-yl]pyridin-2-yl}-N-(tetrahydro-2H-pyr-an-4-yl)benzamide), SM16, IN-1130 (3-((5-(6-methylpyridin-2-yl)-4-(quinoxalin-6-yl)-1H-imidazol-2-yl)methyl-)benzamide), GW6604 (2-phenyl-4-(3-pyridin-2-yl-1H-pyrazol-4-yl)pyridine) and SB-505124 (2-(5-benzo[1,3]dioxol-5-yl-2-tert-butyl-3H-imidazol-4-yl)-6-methylpyridine hydrochloride). Further, while “an ALK5 inhibitor” is not intended to encompass non-specific kinase inhibitors, an “ALK5 inhibitor” should be understood to encompass inhibitors that inhibit ALK4 and/or ALK7 in addition to ALK5, such as, for example, SB-431542. Without intending to limit the scope of the invention, it is believed that ALK5 inhibitors affect the mesenchymal to epithelial conversion/transition (MET) process. TGFβ/activin pathway is a driver for epithelial to mesenchymal transition (EMT). Therefore, inhibiting the TGFβ/activin pathway can facilitate MET (i.e., reprogramming) process.

In another embodiment, the stem cell composition comprises conditioned media from a stem cell culture. The conditioned media contains growth and/or differentiation factors secreted from stem cells. In some embodiments, the stem cells are embryonic stem cells or iPSCs. In other embodiments, the stem cells are hematopoietic stem cells, bone marrow-derived stem cells, mesenchymal stem cells, adipose-derived stem cells and the like.

The stem cell composition may further include one or more differentiation agent(s) for regenerating and/or rejuvenating skin tissues from the stem cell composition. In one embodiment, the differentiation agents include ascorbic acid and/or TGFβ1 for regenerating ectodermal skin tissues. In another embodiment, the differentiation agents include retinoic acid for regenerating keratinocytes and/or bone morphogenetic protein 4 (BMP4) to block neuronal differentiation. In some embodiments, the stem cell composition is a cell-free composition.

### Additional Treatment Steps

In some embodiments, the method further comprises the step of applying a local anesthetic to the target area prior to the introducing step. Examples of local anesthetics include, but are not limited to, procaine, amethocaine, cocaine, lidocaine, prilocalne, bupivacaine, levobupivacaine, ropivacaine, mepivacaine, dibucaine, etidocaine, chloroprocaine, sarapin, benzocaine, tetracaine, pramoxine, oxyprocaine, dyclonine, propoxycaine, chloroxylenol, cinchocaine, dexivacaine, diamocaine, hexylcaine, pyrrocaine, risocaine and rodocaine.

In some embodiments, the method further comprises the step of subjecting the target body surface to one or more additional treatments. Examples of additional treatment include, but are not limited to, galvanic treatment, hyperbaric treatment, electrical stimulation, heat treatment and light treatment. In certain embodiments, the one or more additional treatments are provided concurrently with the CO₂ treatment using the treatment device. In other embodiments, the one or more additional treatments are provided prior to or after the CO₂ treatment.

### CO₂ as a Carrier of a Therapeutic Agent

In some embodiments, CO₂ is administered concurrently with another therapeutic agent. In certain embodiments, CO₂ is administered concurrently with another therapeutic agent and serves as a pharmaceutically acceptable carrier for the therapeutic agent. The therapeutic agent can be in any physical form that is suitable for dispersion in the CO₂ carrier, including, but not limited to, a powder; a liquid; a solution, suspension or emulsion; an oil or a gas. In certain embodiments, the therapeutic agent is formulated for immediate, sustained and/or controlled release of one or more active ingredients. In particular embodiments, the therapeutic agent is a pharmaceutical composition.

In some embodiments, the therapeutic agent comprises the stem cell composition of the instant application. In some embodiments, the stem cell composition comprises a stem cell selected from the group consisting of induced pluripotent stem cells, hematopoietic stem cells, embryonic stem cells, bone marrow-derived stem cells, mesenchymal stem cells, and adipose-derived stem cells. In other embodiments, the stem cell composition is a cell-free composition comprising a conditioned media from a stem cell culture.

In some embodiments, the therapeutic agent comprises one or more iPSC inducing agents as described herein. In some embodiments, the therapeutic agent comprises one or more local anesthetics as described herein. In some embodiments, the therapeutic agent comprises one or more antibiotics. In some embodiments, the therapeutic agent comprises amino acids, peptides and/or proteins. In some embodiments, the therapeutic agent comprises one or more vitamins and/or cofactors. In some further embodiments, the one or more vitamins comprise vitamin A, vitamin B6, vitamin B12, vitamin C, vitamin D, and/or vitamin E. In particular embodiments, the therapeutic agent comprises retinol. In some embodiments, the therapeutic agent comprises one or more toxins. In further embodiments, the one or more toxins include botulinum toxin type A (including, but not limited to, Botox, Dysport and Xeomin) and/or botulinum toxin type B (including, but not limited to, MyoBloc). In some embodiments, the agent comprises collagen, elastin or hyaluronic acid. In some embodiments, the therapeutic agent is an enzyme inhibitor, including, but not limited to, a hyaluronidase or collagenase inhibitor. In some embodiments, the therapeutic agent comprises one or more skin tightening agents. In further embodiments, the skin tightening agents comprise one or more natural skin tightening agents including, but not limited to, plant-based oils, extracts and waxes. In some embodiments, the therapeutic agent comprises copper protein complexes, zinc protein complexes and/or enzyme.

### Administration of CO₂ and Therapeutic Agents

In some embodiments, the effective period of CO₂ treatment is about 30 seconds to about 120 minutes. In some embodiments, the effective period of CO₂ treatment is about 5-120 minutes. In some embodiments, the effective period of time is 30 seconds to 5 min, 1-10 min, 5-30 min, 30-60 min, 60-90 min and 90-120 min.

In some embodiments, the plurality of hollow needles are microneedles having a diameter of about 0.1 mm to about 0.5 mm and a length of about 0.4 mm to about 2.1 mm.

In other embodiments, CO₂ is introduced at a flow rate of 5-360 ml/min.

In some embodiments, the CO₂ treatment step is repeated 1-40 times with an interval of about 24 hours to 3 weeks between any two repeats.

The stem cell composition may be administered before or after administration of the CO₂. In some embodiments, the patient may be treated by: (a) administering a stem cell composition into a target body site in the subject by subcutaneous or topical delivery; (b) affixing a treatment device comprising a plurality of microneedles securely attached to a surface material; (c) applying pressure to the device such that one or more of the plurality of microneedles penetrates the skin of the patient; (d) applying a subcutaneous or intradermal CO₂ administration; and optionally one or more of a galvanic treatment, electrical stimulation or heat treatment; and (e) removing the microneedles from the patient's skin.

As used herein, the terms “needle” and “microneedle” refer to a piercing element suitable for piercing the skin of a patient or subject and passing gas and/or stem cell compositions therethrough in accordance with the methods described herein. The terms are interchangeable unless the context clearly dictates otherwise.

Piercing of the skin surface disrupts the stratum corneum. By only piercing the stratum corneum and/or other layers of the epidermis, the patient typically does not feel pain, trauma (e.g., bleeding and swelling), and/or other discomfort. Subcutaneous or intradermal CO₂ administration allows CO₂ gas to be transported through the disrupted skin to provide the therapeutic benefits. The diameter of the microneedles used to both pierce a patient's skin and to apply subcutaneous or intradermal CO₂ administration in the present application must be of a suitable gauge to allow gas and/or cell compositions to flow there through and at the same time suitable piercing of a variety of skin types and thicknesses, but not so large so as to cause discomfort to the patient when the needles pierce the skin.

The term “plurality of microneedles” means a collection of microneedles arranged for use in the device and methods herein. Such plurality of microneedles must be securably attached to the base material so as to allow suitable insertion into the skin of a patient and also to allow application of other forms of treatment. Examples of suitable materials for use as the microneedles herein can include one or more of metals and metal alloys such as, for example, stainless steel, gold, iron, steel, tin, zinc, copper, platinum, aluminum, germanium, zirconium, titanium and titanium alloys containing molybdenum and chromium, metals or non-metals plated with, gold, rhodium, iridium, titanium, platinum, silver, silver halides, and alloys of these or other metals.

When considered individually, the microneedles used in the present application can have substantially straight or substantially tapered shafts. The diameter of the microneedles can be larger at the base end of the microneedle to taper to a point at the end distal the base. The microneedles can be circular or semi-circular or any other suitable cross-sectional shape. For example, the cross-section of the microneedle can be polygonal (e.g., star-shaped, square, triangular, rectangular), oblong, or another shape. However, whatever shape or length used in the present application, the microneedles must be suitable to provide subcutaneous or intradermal CO₂ administration treatment to a patient in need of such treatment. The microneedles are therefore in fluid communication with a source of CO₂ when the microneedle/base element arrangement is in secure attachment with the treatment device as discussed further herein. When secured to the base material, microneedles can be oriented substantially perpendicular or at an angle thereto. Still further, the microneedles can be oriented substantially perpendicular to the base material. In some aspects, a configuration of microneedles can comprise an arrangement of comprising different microneedle orientations, heights, or other parameters. Generally, the microneedles should have the mechanical strength to resist distortion (such as bending) while being inserted into the skin and while being removed one or more times.

The microneedles can be made from medical-grade steel, such as an acupuncture-type needle, and be from about 0.1 to about 0.5 mm in diameter. Each microneedle can be from about 0.4 to about 2.1 mm in length. In aspect of the invention, each microneedle is from about 0.5 mm and 1.1 mm. Still further, the length of each microneedle can be from about 0.1, 0.3, 0.5, 0.7, 1.0, 1.5, 2.0 or 2.5 mm in length, where any one of these lengths can be used individually or in combination in secure attachment to a base material and to be in fluid communication with a source of CO2 as discussed elsewhere herein.

As noted, microneedles must be securely attached to the base material. In one aspect, the microneedles and a base material to which they are attached comprises a single, disposal one time use unit, otherwise known as a “consumable.” Use of a consumable configuration that securely attaches to a reusable treatment device can facilitate maintenance of a sterile treatment regime, as well as a treatment that is customizable to each patient.

The base material to which the microneedles are securely fastened to provide a microneedle/base element can comprise a rigid material that is sufficiently stiff so as to assist in directing the attached microneedles through a patient's skin. In this regard, the microneedles and base material can be configured as a single unit, such as from stamping of a stainless steel using precision methods that will create needled projections from a flat or substantially flat base material. Use of a metallic material can facilitate application of electrical stimulation and/or heat treatment to a patient in conjunction with subcutaneous or intradermal CO₂ administration as discussed elsewhere herein. In this regard, the microneedles and base materials are comprised of the same material.

Still further, the base material can comprise flexible material to allow the base material to generally conform to the contours of the skin and to adapt to deformations that may occur when the microneedles are inserted. In this regard, microneedles can be securely attached to the base material such as by embedding them into a polymeric material and then applying a suitable adhesive so as to ensure attachment. A flexible surface can facilitates more consistent penetration during use, since penetration can be limited by deviations in the attachment surface.

The depth of CO₂ infusion will vary according to the treatment, for example, the treatment can be from about 2 to about 20 mm. Microneedle length will be substantially equal to the depth of infusion.

When administered subcutaneously, the stem cell composition is injected in one or more target body sites using any suitable subcutaneous delivery methodology known in the art. In one embodiment, the stem cell composition is administered using a cannula with a suitable needle bore size. In some embodiments, the stem cell composition is administered with the treatment device that introduces CO₂ into subcutaneous tissue.

In an exemplary aspect, a medical professional places a treatment device so that the microneedle/base element is in contact or substantially in contact with a location of the skin of a patient. The treatment device is activated by the user, such as by using a plunger, piston or the like, so that at least some of the plurality of microneedles projecting from the base material penetrates the skin of the patient. During the time that such needles are below the surface of the skin, CO₂ is applied from a source and, since the needles are in fluid communication with the source of CO₂, the gas will suitably travel into and below the skin of the patient thereby providing a treatment comprising subcutaneous or intradermal CO₂ administration. Being in fluid communication with a source of CO₂, the plurality of needles suitably allow subcutaneous or intradermal CO₂ administration to a patient in need of such treatment when one or more of the plurality of microneedles is below the surface of a patient's skin.

By way of example, application of subcutaneous or intradermal CO₂ administration for treatments such as skin tightening, rejuvenation, stretch marks and hair regrowth, flow rates for CO₂ gas can be from about 5 to about 300 ml/min. In some embodiments, the CO₂ flow rates are from 50 to about 200 ml/minute, or from about 80 to about 120 ml/min. Still further, CO₂ flow rates can be from about 5, 10, 15, 20, 30, 40, 50, 60, 70, 80, 90, 100, 110, 120, 150, 170 or 200 ml/min, where any value can form an upper or lower endpoint, as appropriate. In some embodiments, the flow rates for CO₂ gas are in the range of 5-10 ml/min, 5-20 ml/min, 5-40 ml/min, 5-80 ml/min, 5-100 ml/min, 5-120 ml/min, 5-150 ml/min, 5-200 ml/min, 5-250 ml/min, 10-20 ml/min, 10-40 ml/min, 10-80 ml/min, 10-100 ml/min, 10-120 ml/min, 10-150 ml/min, 10-200 ml/min, 10-250 ml/min, 10-300 ml/min, 15-20 ml/min, 15-40 ml/min, 15-80 ml/min, 15-100 ml/min, 15-120 ml/min, 15-150 ml/min, 15-200 ml/min, 15-250 ml/min, 15-300 ml/min, 20-40 ml/min, 20-80 ml/min, 20-100 ml/min, 10-120 ml/min, 20-150 ml/min, 20-200 ml/min, 20-250 ml/min, 20-300 ml/min, 40-80 ml/min, 40-100 ml/min, 40-120 ml/min, 40-150 ml/min, 40-200 ml/min, 40-250 ml/min, 40-300 ml/min, 80-100 ml/min, 80-120 ml/min, 80-150 ml/min, 80-200 ml/min, 80-250 ml/min, 80-300 ml/min, 100-120 ml/min, 100-150 ml/min, 100-200 ml/min, 100-250 ml/min, 100-300 ml/min, 120-150 ml/min, 120-200 ml/min, 120-250 ml/min, 120-300 ml/min, 150-200 ml/min, 150-250 ml/min, 150-300 ml/min, 200-250 ml/min, 200-300 ml/min, or 250-300 ml/min. In other embodiments, the CO₂ gas is supplied at a variable flow rate within a single treatment or among multiple sessions of treatments. In some embodiments, a single treatment session comprises a period of high flow rate (e.g., 100-200 ml/min), a period of medium flow rate (e.g., 40-99 ml/min), and a period of low flow rate (e.g., 10-39 ml/min). In some embodiments, a complete treatment regimen comprises one or more sessions at a high flow rate (e.g., 100-200 ml/min), one or more sessions at a medium flow rate (e.g., 40-99 ml/min), and one or more sessions at a low flow rate (e.g., 10-39 ml/min). Time for such treatments can vary from about 30 seconds to about 180 seconds or from about 45 seconds to about 90 seconds. Time for treatment is from about 30, 45, 60, 75, 90, 105, 120, 135, 150, 165 or 180 seconds, where any value can form an upper or lower endpoint, as appropriate.

By way of further example, application of CO₂ by subcutaneous injections for methods of the present application may be conducted at a flow rate from about 40 to about 360 ml/min or from about 90 to about 240 ml/min. In some embodiments, subcutaneous injections can be used for fat reduction in areas such as the chin, arms, knees, thighs, stomach or buttocks.

Still further, CO₂ flow rates for subcutaneous CO₂ and/or stem cell composition treatment can be from about 5, 10, 15, 20, 30, 40, 60, 80, 100, 120, 140, 160, 180, 200, 220, 240, 260, 280, 300, 320, 340 or 360 ml/min, where any value can form an upper or lower endpoint, as appropriate. In some embodiments, the flow rates for CO₂ gas are in the range of 5-10 ml/min, 5-20 ml/min, 5-40 ml/min, 5-80 ml/min, 5-100 ml/min, 5-120 ml/min, 5-150 ml/min, 5-200 ml/min, 5-250 ml/min, 5-300 ml/min, 5-360 ml/min, 10-20 ml/min, 10-40 ml/min, 10-80 ml/min, 10-100 ml/min, 10-120 ml/min, 10-150 ml/min, 10-200 ml/min, 10-250 ml/min, 10-300 ml/min, 10-360 ml/min, 15-20 ml/min, 15-40 ml/min, 15-80 ml/min, 15-100 ml/min, 15-120 ml/min, 15-150 ml/min, 15-200 ml/min, 15-250 ml/min, 15-300 ml/min, 15-360 ml/min, 20-40 ml/min, 20-80 ml/min, 20-100 ml/min, 20-120 ml/min, 20-150 ml/min, 20-200 ml/min, 20-250 ml/min, 20-300 ml/min, 20-360 ml/min, 40-80 ml/min, 40-100 ml/min, 40-120 ml/min, 40-150 ml/min, 40-200 ml/min, 40-250 ml/min, 40-300 ml/min, 40-360 ml/min, 80-100 ml/min, 80-120 ml/min, 80-150 ml/min, 80-200 ml/min, 80-250 ml/min, 80-300 ml/min, 80-360 ml/min, 100-120 ml/min, 100-150 ml/min, 100-200 ml/min, 100-250 ml/min, 100-300 ml/min, 100-360 ml/min, 120-150 ml/min, 120-200 ml/min, 120-250 ml/min, 120-300 ml/min, 120-360 ml/min, 150-200 ml/min, 150-250 ml/min, 150-300 ml/min, 150-360 ml/min, 200-250 ml/min, 200-300 ml/min, 200-360 ml/min, 250-300 ml/min, 250-360 ml/min, or 300-360 ml/min. In some embodiments, a single treatment session comprises a period of high flow rate (e.g., 200-360 ml/min), a period of medium flow rate (e.g., 50-199 ml/min), and a period of low flow rate (e.g., 5-49 ml/min). In some embodiments, a complete treatment regimen comprises one or more sessions at a high flow rate (e.g., 200-360 ml/min), one or more sessions at a medium flow rate (e.g., 50-199 ml/min), and one or more sessions at a low flow rate (e.g., 5-49 ml/min). Treatment times for subcutaneous fact treatment can be from about 1 to about 8 minutes, or from about 2 to about 6 minutes. Still further, the treatment time for subcutaneous injection of CO₂ can be from about 1, 1.5, 2, 3, 3.5, 4, 4.5, 5, 5.5, 6, 6.5, 7.0, 7.5, 8, 10, 15, 20 or 30 minutes, where any value can comprise an upper or lower endpoint as appropriate.

In one aspect, the microneedle/base element and the treatment device can be configured to allow electric current travel into the skin of the patient during the treatment of a patient. The electricity supplied to the disrupted area may also accelerate healing and other benefits that can magnify the therapeutic benefits of CO₂ and/or stem cell composition alone. Alternatively, the microneedles can comprise a conductive material so as to allow application of heat (and/or to heat the CO₂) during a treatment.

In one aspect of the present application, prior to activation of electrical stimulation in the device, the microneedles are inserted into the skin of the patient to disrupt the skin at the desired location(s) thereby, increasing the electric current passage at the selected skin locations to enhance the desirable effect of electric stimulation.

The electrical aspect of the treatment device of the present application can be powered by powered by a power source, such as battery, piezoelectric, electric-mechanical (e.g., a coil magnet), or by a galvanic couple, the disclosure of which is incorporated in its entirety by this reference, so that processes of stratum corneum disruption and electric stimulation are conducted with the same device without the need of changing devices during the treatment.

Still further, the skin treatment device of the present application can comprise suitable materials to provide galvanic action. In this aspect, the microneedles and/or the base material can be made from two dissimilar metals in contact with each other so that they form a galvanic couple, and are therefore capable of generating a galvanic current when the microneedles/base material contacts an electrolyte-containing medium. For example, the base material can comprise a thin zinc sheet, fabricated with the manufacture methods disclosed in U.S. Pat. No. 5,983,136, 6,532,386, 6,050,988, or 6,219,574, (which disclosures are incorporated herein in their entireties by this reference) while another metal (e.g., silver, silver-silver chloride, copper, gold) can be coated on all or part of the areas of one or more of the plurality of microneedles.

During a skin treatment, for example, both metals of the galvanic couple (i.e., zinc and silver-silver chloride) on the microneedle/base element member can be in contact with an electrolyte medium (e.g., a topical composition, or a body fluid such as sweat) and/or the skin to act as a galvanic cell (e.g., of approximately 1 volt) and to generate an electric current, going out from the zinc positive electrode, passing through the electrolyte medium and/or the skin, and returning into the silver-silver chloride negative electrode.

Alternatively, the two metals forming the galvanic couple may be made to contact the third metal (e.g., titanium, or stainless steel) from which can be configured on or within one or more of the microneedle material or base element material. For example, a zinc layer may be coated onto the selective areas of a titanium or stainless steel microneedles member by electric plating, electroless plating, or using a conductive ink including a zinc powder and a polymer binder. Similarly, a silver-silver chloride layer may be coated to other areas of a titanium or stainless steel microneedles. The conductive metallic microneedle serves as a lead to connect the galvanic elements zinc and silver-silver chloride. A galvanic current is generated when both galvanic elements coming into contact with the electrolyte medium and/or the skin during the device application.

In addition, in order to further enhance electrical stimulation and or galvanic action efficacy, the skin of the patient can first be treated with a relatively high concentration of cosmetically acceptable organic solvent, (e.g., glycerin, propylene glycol, or polyethylene glycol), or a non-conductive solute (e.g., low molecular weight sugars, dextrans, or urea).

### Methods of Treatment

In some embodiments, a stem cell composition of the present application is applied to skin in combination with CO₂ to a subject suffering from a disease condition. Examples of the disease conditions include, but are not limited to, psoriasis, Reynaud's, necrotising skin infections, alopecia areata and rheumatoid arthritis.

In other embodiments, a stem cell composition of the present application is applied to skin in combination with CO₂ to rejuvenate skin. As used herein, the term “rejuvenating skin” means increasing the health or condition of skin, improving the appearance of skin and/or decreasing signs of skin aging, for example, decreasing the presence or appearance of wrinkles, fine lines or age spots or increasing the viability of skin cells. The increase or decrease in the foregoing parameters may be at least 5%, 10%, 20%, 50%, 100% or 150% compared to skin untreated in accordance with the present methods and compositions.

In still other embodiments, a stem cell composition of the present application is applied to skin in combination with CO₂ to treat a variety of conditions, including, but not limited to, eczema, seborrhea, vitiligo, lentigo, scleroderma, sunburn, sun damaged skin, estrogen imbalance, hyperpigmentation, hypopigmentation, wrinkles and coarse deep wrinkles, fine lines, skin lines, undereye circles, crevices, bumps, large pores, unevenness, surface roughness, blotchiness, sallowness, loss of skin elasticity, discoloration, yellowing, age spots, freckles, skin atrophy, skin breakout, skin fragility, dryness, chapping, sagginess, thinning, hyperplasia, hyperkeratinization, elastosis, fibrosis, enlarged pores, cellulite formation, bruising, acne vulgaris, cystic acne, acne scars, keloid scars, hypertrophic scars, striae (e.g., stretch marks), dermatitis (e.g., seborrheic dermatitis, nummular dermatitis, contact dermatitis, atopic dermatitis, exfoliative dermatitis, perioral dermatitis, and stasis dermatitis), dermal and epidermal hypoplasia, folliculitis, enlarged pores, ichthyoses (e.g., ichthyosis hystrix, epidermolytic hyperkeratosis, and lamellar ichthyosis), follicular disorders (e.g., pseudofolliculites, senile comedones, nevus comidonicas, and trichostatis spinulosa), benign epithelial tumors (e.g., flat warts, trichoepithelioma, and molluscum contagiosum), perforated dematoses (e.g., elastosis perforans seripiginosa and Kyrles disease), disorders of keratinization (e.g., Dariers disease, keratoderma, hyperkeratosis plantaris, pityriasis rubra pilaris, lichen planus acanthosis nigricans, and psoriasis), pityriasis (e.g., pitiyriasis rosea, pityriasis rosacea and pityriasis rubra), keratoses, impetigo, erysipelas, erythrasma, eczema, senile purpura, excessive sebum (oil) production, sebaceous hyperplasia (enlarged oil glands), viral infections, fungal infections, bacterial infections, spider veins (telangectasia), hirsutism, rosacea, pruritis, calluses, warts and corns.

In some embodiments, the target body surface is suffering from a skin condition selected from the group consisting of acne, psoriasis, skin infections, blemishes, hyperpigmentation, hypopigmentation, alopecia, excessive hair growth, unwanted hair growth, rough skin, dry skin, lax skin, wrinkles, hypervasculatated skin, sebum production disorders, excessive pore appearance, excessive perspiration, hyperhidrosis, tattoo appearance, rashes, scar appearance, pain, itch, burn, inflammation, warts, corns, calluses, edema, ivy/oak poisoning, and bites from insects, spiders, snake, and other animals. In one embodiment, the target body surface is scalp suffering from hair loss. In another embodiment, the target body surface is suffering from alopecia areata. In another embodiment, the target body surface is suffering from diabetic ulcer. In another embodiment, the target body surface is suffering from striae. In yet another embodiment, the target body surface is a scarred body surface.

Another aspect of the present application relates to a method for generating induced pluripotent stem cells (iPSCs) in a subject. The method comprises the steps of subjecting the subject to CO₂ treatment comprising: (1) contacting a target body surface of the subject with a skin treatment device comprising: a plurality of hollow needles attached to a contact surface of a housing; and a CO₂ source in fluid communication with at least one of the plurality of hollow needles; (2) applying pressure to said housing such that one or more of the plurality of hollow needles penetrate an epidermis or an outermost layer of cell in the target body surface; and (3) applying an effective amount of CO₂ to the subject through the plurality of hollow needles for an effective period of time to generate iPSCs. In some embodiments, the iPSCs are generated from autologous hematopoietic stem cells.

In some embodiments, the method further comprises the step of subjecting said target body surface to one or more additional treatments selected from the group consisting of galvanic treatment, electrical stimulation, heat treatment and light treatment.

In some embodiments, the subject is suffering from a condition selected from the group consisting of psoriasis, Reynaud's, necrotising skin infections, alopecia areata and rheumatoid arthritis.

In some embodiments, the CO₂ is introduced at a flow rate of 5-360 ml/min.

In some embodiments, the effective period of CO₂ treatment is about 30 seconds to about 120 minutes. In some embodiments, the effective period of CO₂ treatment is about 5-120 minutes. In some embodiments, the effective period of time is 30 seconds to 5 min, 1-10 min, 5-30 min, 30-60 min, 60-90 min and 90-120 min.

In some embodiments, the CO₂ introducing step is repeated 1-40 times with an interval of about 24 hours to three weeks between any two repeats.

In other embodiments, the method further comprises the step of applying to the target body surface an effective amount of a treatment composition formulized for topical administration. In some embodiments, the treatment composition comprises an effective amount of one or more agents selected from the group consisting of acidic agents, histone deacetylase (HDAC) inhibitors, histone methyl transferase inhibitors and calcium channel activators. In some embodiments, the treatment composition comprises an effective amount of an agent that increases expression of Oct4, Sox2, cMyc and/or Klf4 in a somatic cell or a stem cell.

In some embodiments, the subject is a mammal. In other embodiments, the mammal is a human. In other embodiments, the mammal is a domestic animal, such as a dog, a cat, a monkey, a rat, a mouse, a rabbit, a guinea pig and the like. In other embodiments, the mammal is a farm animal, such as a cow, a horse, a pig, a sheep, a goat, and the like. In yet other embodiments, the mammal is a zoo animal.

Another aspect of the present application relates to a method for treating hair loss in a target area, such as scalp. The method comprises the step of introducing an effective amount of CO₂ into the subcutaneous tissue of the target area for a sufficient period of time. The effective amount of carbon dioxide is introduced into the target area with a plurality of hollow needles that puncture epidermis of the target area and release carbon dioxide at a desired rate within the subcutaneous tissue.

In some embodiments, the CO₂ is introduced into the target area using the skin treatment device and/or the of the skin treatment system of the present application. The plurality of needles in the skin treatment device are selected to have a size and spaced relationship suitable for hair loss treatment.

In some embodiments, the CO₂ is introduced at a flow rate of 50-200 ml/min and for a period of 30 seconds to 120 minutes.

In other embodiments, the introducing step is repeated 4-20 times with an interval of about 24 hours to three weeks between any two repeats.

In some embodiments, the method further include the step of applying a composition comprising a hair growth promoting agent to the target area, wherein the composition is formulated for topical application. Examples of the hair growth promoting agents include, but are not limited to, hair growth factors, minoxidil, finasteride and kopexil, including analogues and derivatives therefrom; cyclosporin 7-thioamide, donepezil hydrochloride, antiandrogenic agents, bimatoprost, Sophora flavescens extract, Serenoa serrulata fruit extract, Serenoa repens extract, licorice extract.

Another aspect of the present application relates to a method for treating a skin condition in a target area. The method comprises the step of introducing an effective amount of CO₂ into the subcutaneous tissue of the target area for a sufficient period of time. The effective amount of carbon dioxide is introduced into the target area with a plurality of hollow needles that puncture epidermis of the target area and release carbon dioxide at a desired rate within the subcutaneous tissue. In some further embodiments, the subcutaneous layer comprises adipose tissue.

In some embodiments, the CO₂ is introduced into the target area using the skin treatment device and/or the of the skin treatment system of the present application. The plurality of needles in the skin treatment device are selected to have a size and spaced relationship suitable for the treatment.

In some embodiments, the CO₂ is introduced at a flow rate of 40-360 ml/min and for a period of 30 seconds to 120 minutes.

In other embodiments, the introducing step is repeated 1-40 times with an interval of about 6 hours to three weeks between any two repeats.

In some embodiments, the method further includes the step of applying a local anesthetic to the target area prior to the introducing step. Examples of local anesthetic include, but are not limited to, of procaine, amethocaine, cocaine, lidocaine, prilocalne, bupivacaine, levobupivacaine, ropivacaine, mepivacaine and dibucaine.

Another aspect of the present application relates to a method for promoting wound healing in a target area, such as scalp. The method comprises the step of introducing an effective amount of CO₂ into the subcutaneous tissue of the target area for a sufficient period of time. The effective amount of carbon dioxide is introduced into the target area with a plurality of hollow needles that puncture epidermis of the target area and release carbon dioxide at a desired rate within the subcutaneous tissue.

In some embodiments, the method further include the step of applying a composition comprising wound healing promoting agent to the target area, wherein the composition is formulated for topical application. Examples of the wound healing promoting agents include, but are not limited to, TGF-related growth factors (TGF-β1, TGF-β2, TGF-133), PDGF-related growth factors (PDGF-M, PDGF-BB, VEGF), FGF-related growth factors (a-FGF, b-FGF, KGF), IGF-related growth factors (IGF-1, IGF-II, insulin), EGF-related growth factors (EGF, HB-EGF, TGFα, amphiregulin, betacellulin), HGF/SF, VEGF, CTGF, TNFα, IL-1, IL-2, IL-6, IL-8, γ-interferon, IL-4, IL-10, matrix metalloproteinases (MMPs; MMP-1, -2, -3, -7, -8, -9, 10, -11, -12, -13, -14, -15, -16, -17, -19, -20, -21, -23A, 23B, -24, -25, -26, -27, -28), tissue inhibitors of metallopeptidases (TIMPs), including TIMP-1, -2, -3 and -4, plasmin, serratiopeptidase (Serratia E-15 protease, also known as serralysin, serratiapeptase, serratia peptidase, serratio peptidase, or serrapeptidase), carboxylates, cipemastat, doxycycline, hydroxamates, marimastat, phosphinyls, tetracyclines, thiols and combinations thereof.

In some embodiments, the CO₂ is introduced into the target area using the skin treatment device and/or the of the skin treatment system of the present application. The plurality of needles in the skin treatment device are selected to have a size and spaced relationship suitable for the treatment.

In some embodiments, the CO₂ is introduced at a flow rate of 5-300 ml/min and for a period of 30 seconds to 120 minutes.

In other embodiments, the introducing step is repeated 5-20 times with an interval of about 24 hours to three weeks between any two repeats, or sessions.

In some embodiments, a session of the method of treating a skin disorder comprises several application steps. A first application step comprises the insertion of needles in a ring a great distance from the skin disorder being treated, for example a distance of between two to four inches. For said first application step, the flow rate of the device is relatively high, for example between 120 and 200 ml/min. Said first application step is followed by at least one intermediate step, wherein the ring of insertion is brought closer to the skin disorder being treated than the first application step or the preceding intermediate step. For each intermediate step, the flow rate is reduced from the application step immediately prior, for example to a flow rate of between 40 and 120 ml/min. The session comprises a final application step to the skin immediately around the periphery of (such as for an open wound) or within (such as for scars or stria) the skin disorder being treated. Said final application step is carried out at a relatively low flow rate, for example at a flow rate of between 5 and 20 ml/min.

In some embodiments, the method further include the step of applying a composition comprising a therapeutic agent to the target area, wherein the composition is formulated for topical application. Examples of the therapeutic agents include, but are not limited to, antimicrobial agents, analgesic and/or non-steroidal anti-inflammatory (NSAID) agents, steroidal/corticosteroidal agents, wound repair agents, anti-cancer agents and skin benefit agents. Exemplary antimicrobial agents include, but are not limited to, anti-bacterial agents (e.g., clindamycin and erythromycin, zithromycin, minocycline, tetracycline, kanamycin, metronidazole, neomycin, bacitracin, polymixin, mafenide acetate, silver sulfadiazine, gentamicin sulfate; anti-fungal agents (terbinafine, itraconazole, micronazole nitrate, thiapendazole, tolnaftate, clotrimazole and griseofulvin caprylyl glycol, triclosan, phenoxyethanol, nystatin or clortrimazole); anti-viral agents (e.g., acyclovir, brivudine, cidofovir, desciclovir, didanosine, famciclovir, 5-fluorouracil, 2-deoxy- and 5-deoxy-5-fluorouridine, ganciclovir, idoxuridine, lamivudine, lobucavir, penciclovir, retrovir, sorivudine, trifluridine, valacyclovir, valganciclovir, vidarabine, zalcitabine, zidovudine, imiquiod, docosanol, brivudin, interferon, famvir, cidofovir, podophyllin, podophyllotoxin); analgesic agents (e.g., aspirin, nonsteroidal anti-inflammatory agents (NSAIDs), salisylates, including salicylic acid, methyl salicylate, olsalazine, sulfasalazine and salsalate; diflunisal, para-aminophenol derivatives, acetanilide, acetaminophen, phenacetin, fenamates, mefenamic acid, meclofenamate, sodium meclofenamate, heteroaryl acetic acid derivatives, tolmetin, ketorolac, diclofenac, propionic acid derivatives, ibuprofen, naproxen sodium, naproxen, fenoprofen, ketoprofen, flurbiprofen, oxaprozin; enolic acids, oxicam derivatives, piroxicam, meloxicam, tenoxicam, ampiroxicam, droxicam, pivoxicam, pyrazolon derivatives, phenylbutazone, oxyphenbutazone, antipyrine, aminopyrine, dipyrone, coxibs, celecoxib, rofecoxib, nabumetone, apazone, indomethacin, sulindac, etodolac, isobutylphenyl propionic acid, lumiracoxib, etoricoxib, parecoxib, valdecoxib, tiracoxib, etodolac, darbufelone, dexketoprofen, aceclofenac, licofelone, bromfenac, loxoprofen, pranoprofen, piroxicam, nimesulide, cizolirine, 3-formylamino-7-methylsulfonylamino-6-phenoxy-4H-1-benzopyran-4-one, meloxicam, lornoxicam, d-indobufen, mofezolac, amtolmetin, pranoprofen, tolfenamic acid, flurbiprofen, suprofen, oxaprozin, zaltoprofen, alminoprofen and tiaprofenic acid); opioid analgesics (e.g., propoxyphene, meperidine, hydromorphone, dihydromorphine, pethidine, hydrocodone, oxycodone, morphine, codeine, and tramodol); NMDA antagonist analgesics (e.g., 2-piperidino-1 alkanol derivatives, ketamine, dextormethorphan, eliprodil, and ifenprodil); skeletal muscle relaxant analgesics (e.g., flexeril, carisoprodol, robaxisal, norgesic, and dantrium); cell differentiating and anti-proliferative agents (e.g., retinoids, such as retinol, retinal, and retinyl esters, such as retinyl acetate, retinyl butyrate, retinyl propionate, retinyl octanoate, retinyl laurate, retinyl palmitate, retinyl oleate, and retinyl linoleate), vitamin D, or vitamin D analogs (calcipotriene)); chemodenervation agents include botulinum type A and/or type B toxins, as well as the serotype C-G toxins; mitochondrial inhibitors (e.g., anthralin (dithranol, chrysarobin, or coal tar)); topical steroids (e.g., clobetasol propionate, betamethasone, betamethasone dipropionate, halobetasol propionate, fluocinonide, diflorasone diacetate, mometasone furoate, halcinonide, desoximetasone, fluticasone propionate, flurandrenolide, triamcinolone acetonide, fluocinolone acetonide, hydrocortisone, hydrocortisone valerate, prednicarbate, desonide, or alclometasone dipropionate); immunosuppressive compounds (e.g., tacrolimus (FK-506)); JAK2 inhibitors (e.g., INCB18424); JAK3 inhibitors (e.g., CP-690,550); parathyroid hormone-related protein (PTHrP) agonists (e.g., PTH(1-34)); and cell adhesion blockers (e.g., pan-selectin antagonist bimosiamose), DNA repair enzymes (e.g., bacteriophage T4 pyrimidine dimer-specific endonuclease, Micrococcus luteus N-glycosylase/AP lyase, Saccharomyces cerevisiae N-glycosylase/apurinic-apyrimidinic lyase, Schizosaccharomyces pombe UV damage endonuclease (UVDE), Chlorella virus isolate PBCV-1 pyrimidine dimer-specific glycosylase, and Anacystis nidulans photolyase); methylxanthines for use in the present application include caffeine (1,3,7-trimethylxanthine), theophylline (1,3-dimethylxanthine), aminophylline, theobromine (3,7-dimethylxanthine), paraxanthine, isobutylmethyl xanthine, butymethylxanthine; salicylic acid, benzoyl peroxide, caffeine, caffeic acid compounds, avobenzone, oxybenzone, octylmethoxycinnamate, titanium dioxide, zinc oxide, dihydroxyacetone, palmitoyl pentapeptide, argireline, interleukins such as IL6 and IL10, growth factors such as EGF and TGF.

Treatment in accordance with the methods of the present application may be localized, such that the target site of a pimple or other blemish, a wrinkle, a razor bumps/ingrown hairs, a herpes sore, a skin infection, an age-spot, or any other skin disorder. Still further, the treatment can be used on larger areas such as the scalp (to enhance hair growth) or the thighs or other areas (for example, to treat cellulite).

In certain embodiments, the treatment method comprises the steps of: administering a stem cell composition into a target body site in the subject by subcutaneous or topical delivery; contacting a target body surface of a subject with a treatment device comprising a plurality of hollow needles attached to a contact surface of a housing and a CO₂ source in fluid communication with at least one of the plurality of hollow needles; applying pressure to the housing such that one or more of the plurality of hollow needles penetrate an epidermis or an outermost layer of cell in the target body surface; skin of the patient; applying a therapeutic amount of CO₂ to the subject through the plurality of hollow needles; and removing the plurality of hollow needles from the target body surface.

In some embodiments, the method further comprises the step of subjecting the target body surface to one or more additional treatments selected from the group consisting of galvanic treatments, electrical stimulations, heat treatments and light treatments. In some other embodiments, the one or more additional treatments are provided concurrently with the subcutaneous or intradermal CO₂ administration using the same treatment device. In some other embodiments, the one or more additional treatments are provided prior to or after the subcutaneous or intradermal CO₂ administration. In some other embodiments, the method further comprises the step of applying to the target body surface an effective amount of a treatment composition formulized for topical administration.

In some embodiments, the plurality of hollow needles are microneedles having a diameter of about 0.1 mm to about 0.5 mm and a length of about 0.4 mm to about 2.1 mm.

The compositions formulated for topical administration may be in the form of a cream, lotion, gel, serum, tonic, emulsion, paste, or spray for topical administration. As used herein, the term “cream” refers to a spreadable composition, typically formulated for application to the skin. Creams typically contain an oil and/or fatty acid based-matrix.

The effective amount of CO₂ depends on the method of treatment. In some embodiments, the effective amount of the CO₂ is defined by the rate of the CO₂ given in the target area and the duration of treatment is a single session. While the effective amount of carbon dioxide may vary from patient to patient, in some embodiments, the CO₂ is introduced into the target area at a flow rate of about 0.1-20 ml/cm²/min, about 0.1-1 ml/cm²/min, about 0.1-2 ml/cm²/min, about 0.1-5 ml/cm²/min, about 0.1-10 ml/cm²/min, about 0.1-15 ml/cm²/min, about 0.5-2 ml/cm²/min, about 0.5-5 ml/cm²/min, about 0.5-10 ml/cm²/min, about 0.5-15 ml/cm²/min, about 0.5-20 ml/cm²/min, about 1-2 ml/cm²/min, about 1-5 ml/cm²/min, about 1-10 ml/cm²/min, about 1-15 ml/cm²/min, about 1-20 ml/cm²/min, about 2-5 ml/cm²/min, about 2-10 ml/cm²/min, about 2-15 ml/cm²/min, about 2-20 ml/cm²/min, about 5-10 ml/cm²/min, about 5-15 ml/cm²/min, about 5-20 ml/cm²/min, about 10-15 ml/cm²/min, about 10-20 ml/cm²/min or about 15-20 ml/cm²/min for a period of about 0.5-60 min, about 0.5-5 min, about 0.5-10 min, about 0.5-20 min, about 0.5-30 min, about 0.5-40 min, about 0.5-50 min, about 2-5 min, about 2-10 min, about 2-20 min, about 2-30 min, about 2-40 min, about 2-50 min, about 2-60 min, about 5-10 min, about 5-20 min, about 5-30 min, about 5-40 min, about 5-50 min, about 5-60 min, about 10-20 min, about 10-30 min, about 10-40 min, about 10-50 min, about 10-60 min, about 20-30 min, about 20-40 min, about 20-50 min, about 20-60 min, about 30-40 min, about 30-50 min, about 30-60 min, about 40-50 min, about 40-60 min or about 50-60 min in a single session.

In some embodiments, the treatment comprises 2-40, 2-30, 2-20, 2-10, 2-5, 4-40, 4-30, 4-20, 4-10, 6-40, 6-30, 6-20, 6-10, 8-40, 8-30, 8-20, 10-40, 10-30, 10-20, 15-40, 15-30, 15-20, 20-40 or 20-30 sessions with an interval of about 6-24, 12-72, 12-48, 12-24 hours or 24 hours to three weeks between each two sessions.

The multi-needle approach decreases the time needed to provide a beneficial amount of CO₂ to a patient in need of such treatment without also reducing the efficacy of such a treatment. Moreover, beneficial CO₂ treatment can be applied to large surfaces in a short period time.

### Skin Treatment Device

Another aspect of the present application relates to reprogramming hematopoietic stem cells in a subject into induced pluripotent stem cells. The method comprises subjecting the subject to a CO₂ treatment that comprises the steps of contacting a target body surface of the subject with a skin treatment device having a plurality of hollow needles attached to a contact surface of a housing and a CO₂ source in fluid communication with at least one of the plurality of hollow needles, applying pressure to the housing such that one or more of the plurality of hollow needles penetrate an epidermis or an outermost layer of cell in the target body surface, introducing an effective amount of CO₂ to the subject through the plurality of hollow needles for an effective period of time, wherein the CO₂ results in a low-pH environment adapted to induce reprogramming of hematopoietic stem cells into induced pluripotent stem cells.

As used herein, the term “hematopoietic stem cells” refers to the blood cells that give rise to all the other blood cells of the myeloid lineage (e.g., monocytes and macrophages, neutrophils, basophils, eosinophils, erythrocytes, megakaryocytes/platelets, dendritic cells) and lymphoid lineage (e.g., T-cells, B-cells, NK-cells). Hematopoietic stem cells include cells with long-term and short-term regeneration capacities and committed multipotent, oligopotent, and unipotent progenitors. Hematopoietic stem cells constitute 1:10.000 of cells in myeloid tissue.

As used herein, the term “low-pH environment adapted to induce reprogramming of hematopoietic stem cells into induced pluripotent stem cells,” refers to a local, lower-than-normal pH environment within the skin tissue of the treated area. In some embodiments, the low-pH environment has a pH value in the range of 4.5-6.5, 4.5-6, 4.5-5.5, 4.5-5, 5-6.5, 5-6, 5-5.5, 5.5-6.5, 5.5-6, 6-6.5, 5.2-6, 5-5.8, 5-5.6, 5-5.4, 5-5.2, 5.2-6, 5.2-5.8, 5.2-5.6, 5.2-5.4, 5.4-6, 5.4-5.8, 5.4-5.6, 5.6-6, 5.6-5.8 and 5.8-6.

Induced pluripotent stem cells, also known as iPS cells or iPSCs, are a type of pluripotent stem cell that can be generated directly from somatic cells. Pluripotent stem cells hold great promise in the field of regenerative medicine. Because they can propagate indefinitely, as well as give rise to every other cell type in the body (such as neurons, heart, pancreatic, and liver cells), they represent a single source of cells that could be used to replace those lost to damage or disease. Strong external stimuli, such as a transient low-pH stressor, are capable of generating pluripotent cells from somatic cells such as splenic CD45+ lymphocytes.

In some embodiments, the effective period of time is 30 seconds to 120 minutes. In some embodiments, the effective period of time is 30 seconds to 5 min, 1-10 min, 5-30 min, 30-60 min, 60-90 min and 90-120 min.

In some embodiments, the plurality of hollow needles are microneedles having a diameter of about 0.1 mm to about 0.5 mm and a length of about 0.4 mm to about 2.1 mm.

In other embodiments, CO₂ is introduced at a flow rate of 5-360 ml/min.

In some embodiments, the CO₂ treatment step is repeated 1-40 times with an interval of about 24 hours to three weeks between any two repeats.

In some embodiments, the CO₂ treatment results in stimulus-triggered acquisition of pluripotency (STAP) in hematopoietic stem cells. In some related embodiments, the CO₂ treatment results in elevated expression of Oct4, Sox2, cMyc and Klf4 in hematopoietic stem cells. In other related embodiments, the CO₂ treatment results in elevated expression of Oct4, Sox2, Nanog and Lin28 in hematopoietic stem cells.

Oct-3/4 (Pou5f1) is one of the family of octamer (“Oct”) transcription factors, and plays a crucial role in maintaining pluripotency. The absence of Oct-3/4 in Oct-3/4+ cells, such as blastomeres and embryonic stem cells, leads to spontaneous trophoblast differentiation, and presence of Oct-3/4 thus gives rise to the pluripotency and differentiation potential of embryonic stem cells. Various other genes in the “Oct” family, including Oct-3/4's close relatives, Oct1 and Oct6, fail to elicit induction, thus demonstrating the exclusiveness of Oct-3/4 to the induction process.

The Sox family of genes is associated with maintaining pluripotency similar to Oct-3/4, although it is associated with multipotent and unipotent stem cells in contrast with Oct-3/4, which is exclusively expressed in pluripotent stem cells. While Sox2 was the initial gene used for induction of pluripotent cells, other genes in the Sox family have been found to work as well in the induction process. Sox1 yields iPS cells with a similar efficiency as Sox2, and genes Sox3, Sox15, and Sox18 also generate iPS cells, although with decreased efficiency.

The Klf family of genes were identified as factors for the generation of iPS cells. Klf2 and Klf4 were found to be factors capable of generating iPS cells, and related genes Klf1 and Klf5 did as well, although with reduced efficiency.

The Myc family of genes are proto-oncogenes implicated in cancer. c-myc is a factor implicated in the generation of mouse and human iPS cells. N-myc and L-myc have been identified to induce pluripotency with an efficiency similar to that of c-myc.

Nanog is a transcription factor critically involved with self-renewal of undifferentiated embryonic stem cells. LIN28 is an mRNA binding protein expressed in embryonic stem cells and embryonic carcinoma cells associated with differentiation and proliferation. Glis1 is a highly promiscuous transcription factor, regulating the expression of numerous genes.

In some embodiments, the method further comprises the step of applying to the target body surface an effective amount of a treatment composition formulized for topical administration. In some related embodiments, the treatment composition comprises an effective amount of an acidic agent. Examples of acidic agents include, but are not limited to, acetic acid, glycolic acid, glutamic acid, butyric acid, and hyaluronic acid. In some other embodiments, the treatment composition comprises an effective amount of a histone deacetylase (HDAC) inhibitor, a histone methyl transferase inhibitor, a calcium channel activator or combinations thereof. In one embodiment, the HDAC inhibitor is a small molecule. In another embodiment, the treatment composition comprises an effective amount of a histone methyl transferase inhibitor and calcium channel activator.

Examples of HDAC inhibitors include, but are not limited to, hydroxamic acids or hydroxamates (such as trichostatin A), cyclic tetrapeptides (such as trapoxin B) and depsipeptides, benzamides, electrophilic ketones and aliphatic acid compounds (such as phenylbutyrate and valproic acid), hydroxamic acids (such as vorinostat, belinostat, LAQ824, panobinostat), benzamides (such as entinostat, CI994, and mocetinostat), abexinostat, SB939, resminostat, givinostat, quisinostat, nicotinamide, CUDC-101, AR-42, CHR-2845, CHR-3996, CG200745, ACY-1215, ME344, sulforaphane, kevetrin, trichostatin A, derivatives of nicotinamide adenine dinucleotide (NAD), dihydrocoumarin, naphthopyranone, 2-hydroxynaphaldehydes, and romidepsin. In one embodiment, the HDAC inhibitor is valproic acid.

Examples of histone methyl transferase inhibitors include, but are not limited to, BIX-01294, chaetocin, 3-deazaneplanocin A hydrochloride, (R)—PFI 2 hydrochloride, SGC 0496, UNC 0224, UNC 0638, UNC 0642 and UNC 0646.

Examples of calcium channel activators include, but are not limited to, (±)-Bay K 8644, (S)-(−)-Bay K 8644, FPL 64176 and nefiracetam.

In some embodiments, the method further comprises the step of applying a local anesthetic to the target area prior to the introducing step.

In some embodiments, the method of further comprises the step of subjecting said target body surface to one or more additional treatments. Examples of additional treatment include, but are not limited to, galvanic treatment, electrical stimulation, heat treatment and light treatment. In certain embodiments, the one or more additional treatments are provided concurrently with the CO₂ treatment using the treatment device. In other embodiments, the one or more additional treatments are provided prior to or after the CO₂ treatment.

In some embodiments, the subject is suffering from a disease condition. Examples of the disease conditions include, but are not limited to, psoriasis, Reynaud's, necrotizing skin infections, alopecia areata and rheumatoid arthritis.

Method of Reprogramming Somatic Cells in a Subject into Induced Pluripotent Stem Cells

Another aspect of the present application relates to a method of reprogramming somatic cells in a subject into induced pluripotent stem cells. The method comprises the step of subjecting the subject to CO₂ treatment that comprises the step of contacting a target body surface of the subject with a skin treatment device comprising: (a) plurality of hollow needles attached to a contact surface of a housing; (b) a CO₂ source in fluid communication with at least one of said plurality of hollow needles; (c) applying pressure to the housing such that one or more of the plurality of hollow needles penetrate an epidermis or an outermost layer of cell in the target body surface; applying an effective amount of CO₂ to the subject through the plurality of hollow needles for an effective period of time, wherein the CO₂ results in a low-pH environment adapted to induce reprogramming of somatic cells into induced pluripotent stem cells.

### Microneedles

As used herein, the terms “needle” and “microneedle” refer to a piercing element suitable for passing gas therethrough that is also suitable to pierce the skin of a patient or subject in accordance with a subcutaneous or intradermal CO₂ administration method. The terms are interchangeable unless the context clearly dictates otherwise.

Piercing of the skin service disrupts the stratum corneum. By only piercing the stratum corneum and/or other layers of the epidermis, the patient typically does not feel pain, trauma (e.g., bleeding and swelling), and/or other discomfort. Subcutaneous or intradermal CO₂ administration allows CO₂ gas to be transported through the disrupted skin to provide the therapeutic benefits. The diameter of the microneedles used to both pierce a patient's skin and to apply subcutaneous or intradermal CO₂ administration in the present application must be of a suitable gauge to allow gas to flow therethrough and at the same time suitable piercing of a variety of skin types and thicknesses, but not so large so as to cause discomfort to the patient when the needles pierce the skin.

The term “plurality of microneedles” means a collection of microneedles arranged for use in the device and methods herein. Such plurality of microneedles must be securably attached to the base material so as to allow suitable insertion into the skin of a patient and also to allow application of other forms of treatment. Examples of suitable materials for use as the microneedles herein can include one or more of metals and metal alloys such as, for example, stainless steel, gold, iron, steel, tin, zinc, copper, platinum, aluminum, germanium, zirconium, titanium and titanium alloys containing molybdenum and chromium, metals or non-metals plated with, gold, rhodium, iridium, titanium, platinum, silver, silver halides, and alloys of these or other metals.

When considered individually, the microneedles used in the present invention can have substantially straight or substantially tapered shafts. The diameter of the microneedles can be larger at the base end of the microneedle to taper to a point at the end distal the base. The microneedles can be circular or semi-circular or any other suitable cross-sectional shape. For example, the cross-section of the microneedle can be polygonal (e.g., star-shaped, square, triangular, rectangular), oblong, or another shape. However, whatever shape or length used in the present invention, the microneedles must be suitable to provide treatment comprising subcutaneous or intradermal CO₂ administration to a patient in need of such treatment. The microneedles are therefore in fluid communication with a source of CO₂ when the microneedle/base element arrangement is in secure attachment with the treatment device as discussed further herein.

When secured to the base material, microneedles can be oriented substantially perpendicular or at an angle thereto. Still further, the microneedles can be oriented substantially perpendicular to the base material. In some aspects, a configuration of microneedles can comprise an arrangement of comprising different microneedle orientations, heights, or other parameters. Generally, the microneedles should have the mechanical strength to resist distortion (such as bending) while being inserted into the skin and while being removed one or more times.

The microneedles can be made from medical-grade steel, such as an acupuncture-type needle, and be from about 0.1 to about 0.5 mm in diameter. Each microneedle can be from about 0.4 to about 2.1 mm in length. In aspect of the invention, each microneedle is from about 0.5 mm and 1.1 mm. Still further, the length of each microneedle can be from about 0.1, 0.3, 0.5, 0.7, 1.0, 1.5, 2.0 or 2.5 mm in length, where any one of these lengths can be used individually or in combination in secure attachment to a base material and to be in fluid communication with a source of CO2 as discussed elsewhere herein.

As noted, microneedles must be securely attached to the base material. In one aspect, the microneedles and a base material to which they are attached comprises a single, disposal one time use unit, otherwise known as a “consumable.” Use of a consumable configuration that securely attaches to a reusable treatment device can facilitate maintenance of a sterile treatment regime, as well as a treatment that is customizable to each patient.

The base material to which the microneedles are securely fastened to provide a microneedle/base element can comprise a rigid material that is sufficiently stiff so as to assist in directing the attached microneedles through a patient's skin. In this regard, the microneedles and base material can be configured as a single unit, such as from stamping of a stainless steel using precision methods that will create needled projections from a flat or substantially flat base material. Use of a metallic material can facilitate application of electrical stimulation and/or heat treatment to a patient in conjunction with treatment comprising subcutaneous or intradermal CO₂ administration as discussed elsewhere herein. In this regard, the microneedles and base materials are comprised of the same material.

Still further, the base material can comprise flexible material to allow the base material to generally conform to the contours of the skin and to adapt to deformations that may occur when the microneedles are inserted. In this regard, microneedles can be securely attached to the base material such as by embedding them into a polymeric material and then applying a suitable adhesive so as to ensure attachment. A flexible surface can facilitates more consistent penetration during use, since penetration can be limited by deviations in the attachment surface.

The depth of CO₂ infusion will vary according to the treatment, for example, the treatment can be from about 2 to about 20 mm. Microneedle length will be substantially equal to the depth of infusion. One aspect of the present application relates to a skin treatment device that is capable of effectively delivering multiple treatment fluids into the cutaneous and/or subcutaneous tissue of a target body area. A benefit of the skin treatment device of the present application is that multiple insertions of the treatment fluid below the skin surface can be effected simultaneously by a practitioner at a controlled flow rate. In some embodiments, the treatment fluid is a gas or a mixture of gases. In other embodiments, the fluid is a liquid, such as a fluid comprising a stem cell composition. In other embodiments, the fluid is a gasified liquid. In a particular embodiment, the fluid is CO₂ gas.

In one embodiment, the skin treatment device of the present application comprises a housing having a contact surface. The contact surface comprises a plurality of hypodermic needles for penetrating the skin and delivering the treatment fluid into or under the skin layer. The plurality of needles on the contact surface are in fluid communication with the treatment fluid source to allow delivery of the treatment fluid from the treatment fluid source into or through the skin. In some embodiments, the plurality of needles protrude between about 0.1 mm and about 10.0 mm from the contact surface. In some further embodiments, the plurality of needles protrude between about 0.15 mm and about 4.0 mm from the contact surface. In further embodiments, the plurality of needles protrude between about 0.3 mm and about 3.0 mm from the contact surface. In still further embodiments, the plurality of needles protrude between about 0.3 mm and about 2.0 mm from the contact surface. In some embodiments, the needles have a length of, or about, 0.1 mm, 0.15 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm, 6.0 mm, 7.0 mm, 8.0 mm, 9.0 mm or 10.0 mm. The needle lengths can control the depth levels and, thus, the exact location of where the treatment fluid will be delivered.

In some embodiments, the needles on the contact surface of the device are all the same length. In other embodiments, the needles on the contact surface of the device are of mixed lengths in order to protrude different depths into or through the body tissue. Needles can be manufactured from stainless steel, and the needle fracture force is hundreds of times greater than the skin insertion force.

In order to cause as little discomfort on the patient as possible during the use of the skin treatment device, the gauge of the needles should be small enough to minimize pain and scarring to the patient. In some embodiments, the size of the needles is sufficient to overcome natural resistance to pierce the stratum corneum. In some embodiments, the needles are between about 26 gauge and about 36 gauge. In some further embodiments, the needles are between about 27 gauge and about 34 gauge. In other further embodiments, the needles are between about 28 gauge and about 33 gauge. In still other further embodiments, the needles are between about 29 gauge and about 32 gauge. In yet further embodiments, the needles are between about 30 gauge and about 32 gauge. In some embodiments, the needles are selected from a gauge of 26, 26s, 27, 28, 29, 30, 31, 32, 33, 34, 35 or 36. In some other embodiments, the needles are microneedles. In some embodiments, the needles on the contact surface of the device are all the same gauge. In other embodiments, the needles on the contact surface of the device are of mixed gauges in order to deliver different volumes of the treatment fluid to different areas of the body tissue.

The size and spacing of the needles may vary depending on the needs of the treatment regimen. In some embodiments, the needles are spaced at about 2, 3, 4, 5, 6, 7, 8, 9, 10 mm apart from each other. The distance between any two needles may be constant or variable. In other embodiments, the needles are arranged in an average density of 1, 2, 3, 4, 5, 6, 7, 8, 9 or 10 needles per cm² of the contact surface. As used herein, the average needle density of the contact surface is defined as the total number of needles divided by the total surface area of the contact surface.

In some embodiments, the contact surface has a total area of about 10-500 cm², about 20-400 cm² or about 40-200 cm². In other embodiments, the contact surface has a total area of about 20 cm², about 40 cm², about 60 cm², about 80 cm², about 100 cm², about 150 cm², about 200 cm², about 250 cm², about 300 cm², about 350 cm², about 400 cm², about 450 cm² or about 500 cm². In some embodiments, the contact surface of the skin treatment device is detachable from the housing and contact surfaces of various sizes and shapes can be attached to the skin treatment device depending on the treatment area and treatment method. In some embodiments, the contact surface has a degree of flexibility that will allow the contact surface to adjust to contours of the body surface to be treated, such as the curvature of the head, thigh etc.

In some embodiments, the contact surface is in the form of a roller. In certain embodiments, the roller has a cylindered shape with a diameter of about 2, 3, 4, 5, 6, 7, 8, 9 or 10 cm and a width (i.e., the distance from side end to side end of the roller) of about 4, 6, 8, 10, 12, 14, 16, 18, 20 cm. In some embodiments, the roller can have a degree of flexibility that will allow the roller to adjust to contours of the body surface to be treated, such as the curvature of the head, thigh etc.

In some embodiments, the housing further comprises a one or more valves that control the flow rate of the treatment fluid. In other embodiments, the housing further comprises a battery. In other embodiment, the house further comprises a control module that controls the fluid flow rate through the one or more valves. In some embodiments, the housing further comprises a fluid inlet to be connected to a fluid source. In other embodiments, the housing comprises a fluid tank located within the housing. In some embodiments, the contact surface is removable and disposable. In other embodiments, the skin treatment device is designed for single use.

FIGS. 1-5 show an embodiment of the skin treatment device 100 designed to introduce CO₂ into a skin tissue of a patient. FIG. 1 depicts the contact surface 110 of a housing 120 (see FIG. 5). In some embodiments, the contact surface 110 is composed of a flexible material such as, but not limited to, polyethylene, polyurethane, silicon or rubber. In other embodiments, the contact surface 110 is composed of a rigid polymer material such as, but not limited to, polystyrene, polycarbonate or polyvinyl chloride. In other embodiments, the contact surface 110 is composed of a rigid polymer material coated with a layer of flexible material. In yet other embodiments, the contact surface 110 is composed of a biodegradable material. The biodegradable material may be selected from any suitable biodegradable material, or from mixtures of two or more thereof. Suitable materials include, but are not limited to, cellulose and cellulosic derivatives, polymers of lactic acid (PLA) and its derivatives, polymers of hydroxyalkanoates (PHAs), biodegradeable copolyesters and polycaprolactones. The biodegradable material may comprise a true biopolymer (PHA or PLA for example), or suitably biodegradable synthetic polymers or suitable mixtures of two or more thereof.

In this non-limiting example, groups of needles are embedded in nodules 112 present on protuberances 114 in the contact surface 110, which is located on the exterior side of a detachable head 122 of the housing 120. Prior to charging the device with the CO₂ gas, the piercing tips of the needles are hidden in the nodules. Because the contact surface 110 is the only part of the skin treatment device 100 that makes physical contact with the body of the patient, in some embodiments, the detachable head 122 of the housing 120 is disposable. In other embodiments, the detachable head 122 of the housing 120 is sterilizable. In some embodiments, the nodules can be used to stimulate, or massage, the skin of the patient prior to injection of the CO₂ gas. When the device is charged with CO₂ gas, the pressure of the CO₂ gas extends the piercing tips of the needles out of the nodules and through the skin of the patient, injecting the CO₂ gas through the skin. In some embodiments, this stimulation or massage of the skin with the nodules serves to mitigate the pain of the needles piercing the skin.

FIG. 2 depicts the interior side 124 of the detachable head 122 of the housing 120. In some embodiments, the interior side 124 of the detachable head 122 comprises chambers through which the CO₂ gas flows to the needles 112 embedded in the contact surface 110. In some embodiments of the skin treatment device 100, the interior side 124 of the detachable head 122 has multiple CO₂ distribution chambers 126 that promotes more even pressure distribution of the CO₂ gas to all of the needles. The CO₂ distribution chambers 126 contains apertures or nodules 125 and are sealed against the base 128 of the housing 120 by rubber or silicon seals 127.

FIG. 3 shows the base 128 of the housing 120. In some embodiments, the base 128 comprises valves 129 that contact apertures or nodules 125 in the interior side 124 of the detachable head 122. In some further embodiments, the valves 129 are pin valves that contact apertures/nodules 125. Opening the valves 129 allows the flow of CO₂ gas into the chambers 126 and through the needles 112 embedded in the contact surface 110 into or through the target skin tissue of the patient.

FIG. 4 shows a control module 130 of the skin treatment device 100. In some embodiments, the control module 130 comprises controls or buttons 132 for opening and closing the valves 129, allowing or stopping the flow of CO₂ gas through to the needles 112. The control module 130 may further comprises one or more lights 134. In some embodiments, at least one light indicates the readiness status of the device for application of CO2. In some embodiments, at least one light indicates the power charge status of the device. In some embodiments, the housing further comprises one or more batteries for powering the device. In some further embodiments, the battery is rechargeable. In alternative further embodiments, the battery is replaceable. In other embodiments, the skin treatment device 100 is powered by a cord that attaches to a separate control device or by an electrical cord to an external power source. The housing 120 further comprises a means for establishing fluid communication of the CO₂ source with the needles that deliver the CO₂ into or through the body tissue. In some embodiments, the CO₂ source is an external tank or reservoir. In alternative embodiments, the CO₂ source is a pressurized CO₂ cartridge that inserts into, or attaches to, the housing 120 of the device. In some embodiments, the CO₂ cartridge is disposable, being replaceable for each new patient. In some embodiments, the controls or buttons 132 or lights 134 are not located on the top of the device, but can be located on the sides, front or back of the device.

FIG. 5 is a side view of an exemplary embodiment of the housing 120. In this non-limiting example, the control module 130 of the housing 120 is knob-shaped to allow easy gripping and manipulation by the gloved hand of a practitioner. In some embodiments, CO₂ gas enters the device from the CO₂ source by way of a tube 140 connected to the device.

In another non-limiting example of the skin treatment device 100, the flat contact surface 110 of the device can be in the form of a roller element. In some embodiments, a plurality of needles are mounted on the roller element, wherein the roller is rotatably mounted on an axle or other suitable configuration of the device that will allow the roller to be rotatable as contemplated herein. The axle or other suitable configuration can include one or a plurality of passageways to allow the CO₂ to be in fluid communication with the needles. Being in fluid communication with a source of CO₂, the plurality of needles suitably allow infusion of CO₂ to a patient in need of such treatment when one or more of the plurality of needles is below the surface of a patient's skin. The number of needle rows that make up the contact surface of the roller element of the present device can vary according to the desired treatment regimen.

In some embodiments, the roller device comprises a plurality of needles permanently attached to the roller device. In some embodiments, the roller element can be disengaged from the device via removable connection. In some embodiments, the roller element is disposable, being replaceable for each new patient. In other embodiments, the roller unit is sterilizable.

In some embodiments, the plurality of needles in the roller element are removable and replaceable. In one embodiment of this removable needle configuration, the needles are integrated into a disposable roller cover. The roller cover can be securely attached to the roller, whereby the roller element is suitably perforated to allow CO₂ to pass into the needles integrated into the roller cover. When the needled roller cover is mounted on the perforated roller element, CO₂ will pass into the skin of the patient when the roller device is placed on a patient's skin. This needled roller cover can enhance safety of the roller device of the present application. When the disposable roller cover is removed from the roller after use, the roller element can be sterilizable to further enhance safety.

In some embodiments, the skin treatment device 100 is attached to a pressured treatment fluid source, such as a CO₂ tank or cartridge. In such embodiments, there is little to no need to include a piston or other type of configuration in order to provide adequate delivery of therapeutic materials under the skin. In some embodiments, the skin treatment device and method of the present application does not comprise a piston or other form of delivery enhancement mechanism. In one embodiment, the present application introduces the treatment fluid into or beneath the skin surface substantially by the pressure from the pressured treatment fluid source.

Another embodiment of a skin treatment device is shown in FIGS. 6-9.

FIG. 6 show one configuration of a treatment device 100 having a handle portion 202. At a first end of handle portion 202, a hose element 204 is attached to a source of CO₂ gas (not shown). As an optional feature, a regulator 206 can be included on treatment device 100. A switch 208 can facilitate operation. In use, end piece 210 is attachable to microneedle/base element 212. Microneedle 214 and base material 216 are also shown.

Referring to FIG. 7, microneedle/base element 212 is shown in contact with patient's skin 300 and in penetration to an interior skin portion 302. The microneedles 214 have a hollow end 218 that will fill with CO₂ gas (not shown) in a penetration area 304.

Referring to FIG. 8, an exemplary configuration of a microneedle/base element 220 is shown. An area of base material 216 is provided with a plurality of microneedles 214. Referring to FIG. 9, another exemplary configuration of a microneedle/base element 222 is shown. An area of base material 216 is provided with a plurality of microneedles 214. The shape of the base element is not limited to the exemplified shapes. The present application envisions any shape of base element that is suitable for carrying out the methods disclosed in the present application. The shape may be determined by the contours of the area of the body to be treated or by the need to avoid a particular bodily feature.

In another embodiment, the device of the present application comprises a plurality of interchangeable head units with rings of microneedles of varying diameters. For example, the device comprises a first interchangeable head with a ring, or rings, of microneedles with a large diameter so that when the ring(s) is/are centered on the skin disorder being treated, the ring(s) is/are at the greatest distance from the skin disorder. The device further comprises one or more interchangeable heads having ring(s) of smaller diameter than the ring(s) on said first interchangeable head, thereby bringing the ring(s) closer to the skin disorder being treated.

In some embodiments, the device of the present application is capable of delivering both CO₂ and the stem cell composition of the present application.

### Skin Treatment System

Another aspect of the present application relates to a skin treatment system comprising a self-contained treatment fluid source, a pressure regulator for regulating the pressure of the treatment fluid, an filter for filtering out contaminants such as bacteria, viruses and other gaseous impurities in the treatment fluid, a heating system for raising the temperature of the treatment fluid, a flow regulator for controlling the flow of the treatment fluid, and a skin treatment device for delivering the treatment fluid into or beneath the skin of a patient. In some embodiments, the treatment fluid is CO₂ gas. In a further embodiment, the CO₂ gas is medical grade CO₂ gas, thereby reducing or eliminating the need for a filter.

In an exemplary use of the skin treatment device of the present application, the contact surface or the roller of the skin treatment device is directed over the skin of a patient so that at least some of the plurality of needles projecting from the contact surface penetrate the skin of the patient. During the time that such needles are below the surface of the skin, the treatment fluid, such as CO₂, is applied from the treatment fluid source. Since the needles are in fluid communication with the treatment fluid source, the treatment fluid will suitably travel into and below the skin of the patient in a desired amount over a desired period time to provide the desired treatment. Depending the length of the needles, the treatment can be conveyed at various distances below the patient's skin surface. In the case of a roller type contact surface, the frequency of the movement of the roller over the skin determines the number of puncture channels, which can be controlled specifically and thus also the degree to which the treatment fluid can penetrate the patient's skin.

Kit

In one aspect, a treatment device of the present application, the microneedle/base element and one or more companion products are packaged together as a kit. Exemplary items in the kit may include, but are not limited to, the device including a pre-determined number of disposable or replaceable microneedle/base element configurations, a reusable treatment device for application of and one or more additional treatments of electrical stimulation, galvanic action or heat therapy, a stem cell composition and/or topical treatment composition in a suitable container/dispenser (such as a tube, a bottle, a pump, a jar, a dropper, a or unit-dose dispenser) to be used before, during, or after the device application. Additionally, the kit may also contain a cleansing product to be used to sanitize/sterilize the skin prior to the device application. The kit may also include a film forming composition or bandage to be used after treatment to protect the treated skin site and to enhance the therapeutic efficacies for the treated skin.

The present invention is further illustrated by the following examples which should not be construed as limiting. The contents of all references, patents and published patent applications cited throughout this application, as well as the Figures and Tables, are incorporated herein by reference.

### Example 1

### Treatment of Alopecia Areata

A female patient presented with Alopecia Areata and had been suffering with this condition for over seven years. The patient had been to 14 different specialists in search of an adequate treatment, but was relegated to wearing a wig. No treatments, including the topical application of Minoxidil and hair growth factors had produced satisfactory relief from the condition. The patient was subjected to once-weekly treatment comprising subcutaneous CO₂ administration and was administered in a clinical setting. Warmed CO₂ was administered at a flow rate of 80 ml/min. Additionally, the patient was instructed to continue once daily topical application of Minoxidil and hair growth factor.

After ten clinical sessions in two and a half months, the patient exhibited significant hair ingrowth into the bald patches. The clinical treatment protocol was reduced to subcutaneous CO₂ administration two times per month. Following 20 sessions of clinical subcutaneous CO₂ administration in about six months, the patient exhibited hair growth throughout the bald patches.

### Example 2

### Treatment of Male-Pattern Baldness

An adult male patient presented with hair loss around the whorl. The patient was clinically treated with subcutaneous CO₂ administration and within three months significant regrowth of hair around the whorl was seen. After 5 months of subcutaneous or intradermal CO₂ administration, the patient experienced full ingrowth of normal-textured hair around the whorl.

### Example 3

### Treatment of Generalized Hair Thinning

A male patient presented with generalized thinning of the hair across the entire crown of his scalp. The patient was subjected to once-monthly clinical subcutaneous CO₂ administration. At three months, the patient exhibited substantial in-filling of the thinned region. The treatment protocol was changed to twice-monthly subcutaneous CO₂ administration and at five months the patient showed significant coverage of the crow region with normal-textured hair.

### Example 4

### Treatment of Diabetic Ulcers

Ten insulin-dependent diabetic patients with chronic lower extremity wounds were referred for CO₂ transdermal treatment. The control group consisted of five of the patients, three who were claustrophobic and two who refused CO₂ insufflation treatments because of logistic reasons. Five patients underwent 30 CO₂ insufflation treatments in the problem wound protocol (30 min/day, 5 days/week). All patients were evaluated with transcutaneous oxygen measurements and had an initial surgical debridement of the wound.

An exemplary protocol for a CO₂ insufflation treatment of an ulcerated wound comprises several steps of CO₂ application. A first application step comprises inserting microneedles in a ring of large radius around the wound, for example about two inches from the edges of the wound. CO₂ gas is insufflated at a high flow rate, for example between 120 and 200 ml/min, more particularly about 150 ml/min. This is followed by a second application in a smaller ring, about one inch from the edges of the wound. CO₂ gas is insufflated at a lower flow rate, for example between 50 and 100 ml/min, more particularly about 80 ml/min. This is followed by a third application in a still smaller ring, about one/half inch from the edges of the wound. CO₂ gas is insufflated at a still lower flow rate, for example between 30 and 50 ml/min, more particularly about 40 ml/min. A last application step is made in the skin immediately around the periphery of the wound using a single microneedle applicator using a very low flow rate of between about 5 and 25 ml/min, more particularly about 15-20 ml/min.

Weekly tracings of the wound surface area were made by a nurse or resident who was blinded to the group assignment. At the end of 7 weeks, the mean wound area expressed as a percentage of pretreatment baseline area was compared between groups (analysis of variance, Duncan's post hoc). No significant differences were noted between groups with respect to age, gender, baseline wound area, wound site O₂ tension, or presence of osteomyelitis. At the completion of each of the 7-wk treatment periods, a significantly greater reduction in wound surface area was noted in the CO₂ vs. the control group (P<0.05).

A 65 year old male diabetic patient with hypertension and occlusive arterial disease in the lower limbs (ankle-brachial index 0.4) presented with a three-month post-traumatic wound in the metatarsal area of the left foot. Prior to carboxytherapy, the patient was treated with antibiotics and extensive necrotic exeresis with no revascularization procedure. After six applications of treatment comprising subcutaneous CO₂ administration, the wound has begun to close and new skin is forming over the wound after 12 applications of subcutaneous CO₂ administration. Following 16 applications of subcutaneous CO₂ administration, there was a substantial reduction in size of the wound, with the remainder being covered over by a scab. The wound was fully covered over by new skin growth after 20 subcutaneous CO₂ administrations.

A 51 year old male diabetic patient having chronic venous insufficiency in his left lower limb with secondary deep venous thrombosis. The patient presented with open ulcers (clinical, aetiological, anatomical and pathological elements (CEAP) classification C6) that had failed to respond to conventional curative and compressive therapies for the previous 12 months. The patient was started on a course of subcutaneous CO₂ administration, along with maintenance of the conventional therapies already in use. The wound begun to close after four applications of carboxytherapy treatment, and was covered by a scab after eight carboxytherapy applications. There was a reduction in size of the wound after 12 subcutaneous CO₂ administrations and the wound was substantially closed after 16 subcutaneous CO₂ administration sessions.

### Example 5

### Treatment of Striae

Striae, commonly known as stretch marks, can appear when there is rapid stretching of the skin. They are often associated with the abdominal enlargement of pregnancy. They also can be found in children who have become rapidly obese or may occur during the rapid growth of puberty in males and females. Striae are most commonly located on the breasts, hips, thighs, buttocks, abdomen, and flank.

A female patient presented with striae on the buttocks and was treated with a course of once/monthly treatments comprising subcutaneous CO₂ administration at an administration rate of 80 ml/min. Following six subcutaneous CO₂ administration sessions, the appearance of the striae was significantly diminished.

### Example 6

### Treatment of Surgical Scars

Scars are a natural result of some kind of injury to the skin. They can occur because of surgery, accidental injury, acne or infection. As skin heals, the area can become thickened, raised and discolored, resulting in a permanent scar. Some scars fade with time, but most remain visible. Even those that fade may take years to do so. In some instances, scar tissue may cause physical discomfort. In others, visible scars may cause embarrassment or emotional discomfort for a patient, for example, such as scars left after some types of reconstructive surgery.

A patient presented with scars remaining near the ear following a facelift procedure. Following 4 subcutaneous CO₂ treatments the visibility of the scars was greatly diminished.

A female patient presented with raised, darkened, keloid scars on the breasts and abdomen following reconstructive surgery. The patient was treated with a course of once/monthly subcutaneous CO₂ treatment at a flow rate of 80 ml/hr. Following six treatments, the scars were no longer raised and had lightened to more closely approximate the patient's natural skin tone.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A method for treating skin-related disorder or improving the condition of skin in a subject, comprising:
administering a stem cell composition into a target body site in the subject by subcutaneous, intradermal or topical delivery, and
administering CO₂ to the target body site by subcutaneous or intradermal administration,
wherein the CO₂ and the stem cell composition are provided in an amount effective for treating said a skin-related disorder or improving the condition of skin.

2. The method of claim 1, wherein said stem cell composition comprises a stem cell preparation, a composition comprising one or more iPSC inducing agents or conditioned media obtained from a stem cell culture.

3. The method of claim 1, wherein said stem cell composition comprises a stem cell preparation comprising a stem cell selected from the group consisting of hematopoietic stem cells, embryonic stem cells, bone marrow-derived stem cells, mesenchymal stem cells, and adipose-derived stem cells.

4. The method of claim 1, wherein said stem cell composition comprises an induced pluripotent stem cell (iPSC) or an iPSC inducing agent.

5. The method of claim 4, wherein said iPSC is derived from a fibroblast cell or an autologous cell.

6. The method of claim 4, wherein said iPSC is derived from a cell selected from the group consisting of cells isolated from peripheral blood, bone marrow and umbilical cord blood.

7. The method of claim 4, wherein said one or more iPSC inducing agents comprise at least one member selected from the group consisting of Oct 3/4, Sox2, Klf4, c-Myc, Lin28 and Nanog.

8. The method of claim 4, wherein said one or more iPSC inducing agents comprise at least one member that increases the expression of Oct4, Sox2, Klf4, c-Myc, Lin28 and/or Nanog.

9. The method of claim 4, wherein said one or more iPSC inducing agents comprise one or more members selected from the group consisting of H3K9 methylation inhibitors, H3K demethylation promotors, HDAC inhibitors, L-type Ca channel agonists, cAMP pathway activators, DNA methyltransferase (DNMT) inhibitors, nuclear receptor ligands, GSK3 inhibitors, MEK inhibitors, TGFβ receptor/ALK5 inhibitors, Erk inhibitors and combinations thereof.

10. The method of claim 1, wherein said stem cell composition comprises a stem cell extract, cell-free components secreted from a stem cell culture, or one or more differentiation agents.

11. The method of claim 10, wherein said cell-free components are recovered from an embryonic stem cell culture or an iPSC culture.

12. The method of claim 10, wherein said cell-free components are recovered from a culture of cells selected from the group consisting of hematopoietic stem cells, bone marrow-derived stem cells, mesenchymal stem cells and adipose-derived stem cells.

13. The method of claim 10, wherein said stem cell extract comprises an embryonic stem cell extract or an iPSC extract, wherein said cell extract prepared from a culture of cells selected from the group consisting of hematopoietic stem cells, bone marrow-derived stem cells, mesenchymal stem cells and adipose-derived stem cells.

14. The method of claim 10, wherein said one or more differentiation agents is selected from a group consisting of ascorbic acid, TGFβ1, retinoic acid, and bone morphogenetic protein 4.

15. The method of claim 1, further comprising the step of subjecting said target body surface to one or more additional treatments selected from the group consisting of galvanic treatment, electrical stimulation, heat treatment and light treatment.

16. The method of claim 15, wherein said one or more additional treatments are provided concurrently with said CO₂ treatment using said treatment device.

17. The method of claim 15, wherein said one or more additional treatments are provided prior to or after said CO₂ treatment.

18. The method of claim 1, wherein said device for introducing CO₂ into the subcutaneous tissue comprises a plurality of hollow needles having a diameter of about 0.1 mm to about 0.5 mm and a length of about 0.4 mm to about 2.1 mm.

19. The method of claim 1, wherein said subject has a skin-related disorder selected from the group consisting of psoriasis, Reynaud's disease, a necrotising skin infection, alopecia areata and rheumatoid arthritis.

20. A method for generating induced pluripotent stem cells (iPSCs) in a subject, comprising:
subjecting said subject to a CO₂ treatment comprising:
contacting a target body surface of said subject with a skin treatment device comprising a plurality of hollow needles attached to a contact surface of a housing; and a CO₂ source in fluid communication with at least one of said plurality of hollow needles;
applying pressure to said housing such that one or more of the plurality of hollow needles penetrate an epidermis or an outermost layer of cell in said target body surface; and,
applying an effective amount of CO₂ to said subject through said plurality of hollow needles for an effective period of time to generate iPSCs.

21. The method of claim 20, further comprising the step of subjecting said target body surface to one or more additional treatments selected from the group consisting of galvanic treatment, electrical stimulation, heat treatment and light treatment.

22. The method of claim 20, wherein said iPSCs are generated from autologous hematopoietic stem cells.

23. The method of claim 20, wherein said CO₂ is introduced at a flow rate of 5-360 ml/min.

24. The method of claim 20, wherein said effective period of time is 30 seconds to 120 minutes.

25. The method of claim 20, wherein said introducing step is repeated 1-40 times with an interval of about 24 hours to 3 weeks between any two repeats.

26. The method of claim 20, further comprising the step of applying to said target body surface an effective amount of a treatment composition formulized for topical administration.

27. The method of claim 20, wherein said treatment composition is selected from a group consisting of an effective amount of an acidic agent, a histone deacetylase (HDAC) inhibitor, a histone methyl transferase inhibitor, calcium channel activator and an effective amount of an agent that increases expression of Oct4, Sox2, cMyc and/or Klf4 in a somatic cell or a stem cell.

28. A method for treating a disorder in a subject, comprising:
administering a stem cell composition into a target body site in the subject by subcutaneous, intradermal or topical delivery, and
administering CO₂ to the target body site by subcutaneous or intradermal administration,
wherein the CO₂ and the stem cell composition are provided in an amount effective for treating said disorder, wherein said disorder is selected from the group consisting of psoriasis, Reynaud's disease, a necrotising skin infection, alopecia areata and rheumatoid arthritis.

29. A method for treating a skin-related condition in a subject, comprising subjecting the subject to CO₂ treatment that comprises the step of:
contacting a target body surface of the subject with a skin treatment device comprising: plurality of hollow needles attached to a contact surface of a housing; and a CO₂ source in fluid communication with at least one of said plurality of hollow needles;
applying pressure to the housing such that one or more of the plurality of hollow needles penetrate an epidermis or an outermost layer of cell in the target body surface; and
applying an effective amount of CO₂ to the subject through the plurality of hollow needles for an effective period of time to generate a low-pH environment adapted to induce reprogramming of somatic cells into induced pluripotent stem cells.

30. The method of claim 29, wherein said skin-related condition is selected from the group consisting of psoriasis, Reynaud's disease, a necrotising skin infection, alopecia areata and rheumatoid arthritis.
